(19) United States
(12) Patent Application Publication
(10) Pub. No.: US 2014/0315202 A1
(43) Pub. Date: Oct. 23, 2014

(54) METHOD FOR ASSEMBLING MULTIPLE TARGET LOCI TO SINGLE NUCLEIC ACID SEQUENCE

(71) Applicants: Du Hee Bang, Seoul (KR); Hyo Jun Han, Seoul (KR)

(72) Inventors: Du Hee Bang, Seoul (KR); Hyo Jun Han, Seoul (KR)

(73) Assignee: INDUSTRY-ACADEMIC-COOPERATION FOUNDATION, YONSEI UNIVERSITY, Seoul (KR)

(21) Appl. No.: 14/239,785

(22) PCT Filed: Mar. 13, 2012
(86) PCT No.: PCT/KR2012/001808

§ 371 Date: Feb. 20, 2014

(30) Foreign Application Priority Data
Mar. 16, 2011 (KR) 10-2011-0023184

(51) Int. Cl. C12Q 1/68; C12P 19/34

(52) U.S. Cl. CPC C12Q 1/686; C12P 19/34; C12Q 1/6869
USPC 435/6.11; 435/91.2; 435/6.12

(57) ABSTRACT

A method for assembling multiple target loci to a single assembled nucleic acid sequence involves assembling the multiple target loci to a single assembled nucleic acid sequence through two polymerase chain reactions (PCRs). A pair of primers for a primary amplification include a target-specific sequence and a 5′-flanking assembly spacer sequence. A primary amplified product amplified by the pair of primers for a primary amplification is assembled to a single shortened nucleic acid sequence in a convenient and easy manner through a set of primers for a secondary amplification, thus enabling the simultaneous detection of multiple target loci. Accordingly, the method and a kit of the present invention may simultaneously detect and analyze multiple variabilities in DNA sequences of a sample, thus remarkably reducing sequencing costs for detecting variabilities, and providing a critical approach and means for achieving the concept of customized medicine.

### CROSS REFERENCE TO RELATED APPLICATIONS AND CLAIM OF PRIORITY

This patent application is a National Phase application under 35 U.S.C. §371 of International Application No. PCT/KR2012/001808, filed 13 Mar. 2012, which claims priority to Korean Patent Application No. 10-2011-0023184, filed 16 Mar. 2011, entire contents of which are incorporated herein by reference.

### TECHNICAL FIELD

The present invention relates to a method for assembling multiple target loci into a single assembled nucleic acid sequence and a method for simultaneously detecting multiple target loci using the same.

### DESCRIPTION OF THE RELATED ART

The PCR-based amplification of a genomic locus has provided the simplest protocol for targeted sequencing (1-3). Multiple loci can be targeted by adding several primer pairs to generate amplicons (4, 5). Recently developed methods allow us to capture desired genomic loci selectively prior to sequencing (6-13). For example, RainDance, Inc. has produced a micro-fluidic platform to amplify thousands of amplicons simultaneously (14). Other large-scale target-enrichment methods use ‘molecular inversion probes’ (MIP) (10-13) and the hybrid capture approach (6-9). These target-enrichment methods have significantly increased the multiplexity in the capture of target DNA (2). By combining these target-enrichment strategies with high-throughput DNA sequencing technology, researchers have been able to reduce the costs of sequencing dramatically.

However, to the best of our knowledge, no methodology can achieve multiplex target sequencing using a single sequencing read. For example, when multiple target loci cannot be amplified at once using conventional PCR, multiple target loci must be amplified separately and sequenced multiple times (4).

In this connection, there has been urgently demanded in the art a method capable of obtaining a variety of target loci in a single sequencing read.

Throughout this application, various publications and patents are referred and citations are provided in parentheses. The disclosures of these publications and patents in their entities are hereby incorporated by references into this application in order to fully describe this invention and the state of the art to which this invention pertains.

### SUMMARY OF THE INVENTION

The present inventors have endeavored to develop new methods for detecting multiple target loci more efficiently and accurately. As a result, the present inventors have found that primary amplification products, which are obtained by amplification using a primary amplification primer set including at least two primer pairs each of which is hybridized with upstream and downstream regions of a target locus and includes a flanking region of the target locus, are conveniently and easily assembled into a single shortened nucleic acid sequence by using a secondary amplification set, so that multiple target loci can be simultaneously detected, and the completed the present invention.

Accordingly, an aspect of the present invention is to provide a method for assembling multiple target loci into a single shortened nucleic acid sequence.

Another aspect of the present invention is to provide a method for simultaneously detecting multiple target loci.

Still another aspect of the present invention is to provide a kit for detecting multiple target loci.

Other purposes and advantages of the present invention will become clarified by the following detailed description of invention, claims, and drawings.

### BRIEF DESCRIPTION OF THE DRAWINGS

FIG. 1 is schematic representation of the mTAS (multiple target loci assembly sequencing) method.

FIG. 2 represents gel data from the first PCR of the mTAS experiments.

FIG. 3 shows agarose gel data from the second PCR of mTAS target amplification of 20 different sets of human genomic loci (79+3 loci in cancer patient). Red triangles indicate the desired target amplicon sizes.

FIG. 4 is summary of the mTAS target sequencing data of 20 different sets of human genomic loci. The mTAS experiments were repeated three times (shown in green, blue, and red bars, respectively). The horizontal axis indicates the rate of the desired target sequences from the target assembly sequences based on the Sanger sequencing result (Tables 12-16).

FIG. 5 is gel data from mTAS experiments for EGFR mutations present at three different exons from lung cancer patients. Agarose gel data show mTAS target results for EGFR mutation from normal tissue (A) and tumor tissue (B). Red triangles indicate the desired target amplicon sizes. For eight tumor and normal tissue samples, ‘T’ indicates tumor tissue and ‘N’ indicates normal tissue. Panel C is direct Sanger sequencing result from mTAS for EGFR mutation. Patients 1, 2 and 8 had Exon 21 Leu858Arg mutation. Patients 3 and 4 had Exon 19 deletion (5′-GAATTAAGAGAAGCA-3′) (SEQ ID NO: 216) mutation. Patients 5, 6 and 7 had lung cancer from a type of cancer mutation other than EGFR.

### DETAILED DESCRIPTION OF THIS INVENTION

In accordance with an aspect of the present invention, there is provided a method for assembling multiple target loci into a single shortened nucleic acid sequence, the method including: (a) obtaining a target nucleic acid molecule including multiple target loci including at least two target loci on one molecule thereof; (b) obtaining primary amplification products by primary amplification of the target nucleic molecule using a primary amplification primer set including at least two primer pairs for being hybridized with upstream and downstream regions of the at least two target loci and amplifying flanking regions of the at least two target loci, wherein the at least two primer pairs each having a forward primer and a reverse primer and the at least two primer pairs include a first primer pair for amplifying a first target locus which is located relatively in the 5′ direction and a second primer pair for amplifying a second target locus which is located in the 3′ direction of the first target locus; wherein a reverse primer of the first primer pair includes (i) a target hybridization nucleotide sequence that is complementary to a downstream region of the first target locus and (ii) an overlapping sequence that is non-complementary to the target nucleic acid molecule but complementary to a forward primer of the second primer pair; and wherein the forward primer of the second primer pair includes (i) a target hybridization nucleotide sequence that is complementary to an upstream region of the second target locus and (ii) an overlapping sequence that is non-complementary to the target nucleic acid molecule but complementary to the reverse primer of the first primer pair; and (c) obtaining secondary amplification products by secondary amplification using a secondary amplification primer set and the primary amplification products, the secondary amplification primer set including a primer that is complementary to a 5′ end region formed when the primary amplification products are arranged in the 5′ to 3′ direction and a primer that is complementary to a 3′ end region of the sequence, wherein the secondary amplification products constitute a nucleic acid sequence in which the at least two target loci are located adjacent to each other, the nucleic acid being extended to have a greater length than the target nucleic acid molecule used in step (a).

The present inventors endeavored to develop new methods for detecting multiple target loci more efficiently and accurately. As a result, the present inventors found that primary amplification products, which are obtained by amplification using a primary amplification primer set including at least two primer pairs each of which is hybridized with upstream and downstream regions of a target locus and includes a flanking region of the target locus, are conveniently and easily assembled into a single shortened nucleic acid sequence through a secondary amplification set, so that multiple target loci can be simultaneously detected.

The present invention provides a method for assembling a single shortened nucleic acid sequence including multiple target loci by performing a primary polymerase chain reaction (PCR) using a primary amplification primer set and a secondary PCR using primary amplification products and a secondary amplification primer set.

According to the method of the present invention, nucleotide sequences of multiple target loci can be simultaneously detected by performing a PCR of a target nucleic acid molecule including at least two target loci.

As used herein, the term “nucleotide” refers to dioxyribonucleotide or ribonucleotide existing in a single-strand type or a double-strand type, and includes analogs of naturally occurring nucleotides unless otherwise particularly specified (Scheit, Nucleotide Analogs, John Wiley, New York (1980); Uhlman and Peyman, Chemical Reviews, 90:543-584 (1990)).

According to a preferred embodiment of the present invention, gene amplification of the present invention is performed by a polymerase chain reaction (PCR). According to a preferred embodiment of the present invention, the primer of the present invention is used in gen amplification reactions.

As used herein, the term “primer” refers to an oligonucleotide, and may act as an initiation point in the conditions where the synthesis of the primer extension products complementary to a nucleic acid chain (template) is induced, that is, the presence of polymerases such as nucleotide and DNA polymerases and appropriate temperature and pH. Preferably, the primer is dioxyribonucleotide, and has a single chain. The primer used herein may include naturally occurring dNMP (that is, dAMP, dGMP, dCMP, and dTMP), modified nucleotides, or non-naturally occurring nucleotides. Also, the primer may include ribonucleotide.

The primer needs to be long enough to prime the synthesis of extension products in the presence of polymerases (for example, DNA polymerase). The appropriate length of the primer varies depending on several factors, such as temperature, field of application, and primer source, but the primer has generally 15˜30 nucleotides. A short primer molecule generally requires a low temperature in order to form a sufficiently stable hybridization complex together with a template. According to a preferable embodiment of the present invention, the primer of the present invention is constructed by using a computer program, Perl-mTAS.

As used herein, the term “annealing” or “priming” refers to the apposition of oligodeoxynucleotide or nucleic acid to a template nucleic acid. The apposition enables the polymerase to polymerize nucleotides into a nucleic acid molecule which is complementary to the template nucleic acid or a portion thereof. As used herein, the term “hybridization” refers to the formation of a duplex structure by pairing of complementary nucleotide sequences of two single-strand nucleic acids. The hybridization may occur when complementarity between single-strand nucleic acid sequences is perfect (perfect match) or some mismatch bases are present. The degree of complementarity required for hybridization may be changed depending on hybridization reaction conditions, and may be controlled by particularly, the temperature. As used herein, the term “annealing” and “hybridization” are not substantially differentiated from each other, and thus are used together.

According to a preferred embodiment of the present invention, the primer used herein is specifically constructed in the PCR step (for example, a primary PCR and a secondary PCR) and then used.

More specifically, the primer pair for primary amplification (a forward primer and a reverse primer) consists of a target-specific sequence (target hybridization nucleotide sequence) and a 5′-flaking assembly spacer sequence (overlapping sequence). As used herein, the term “target-specific sequence (target hybridization nucleotide sequence)” is a sequence complementary to a target locus to be amplified, and located in the 3′-direction within the primer. In addition, as used herein, the term “5′-flaking assembly spacer sequence (overlapping sequence)” is a sequence that is non-complementary to the target loci to be amplified, and located in the 5′-direction within the primer.

The overlapping sequence functions as an overlapping region that enables specific annealing between mutually independent target loci. For example, when two multiple target loci are assembled by using two primer pairs including a first primer pair for amplifying a first target locus which is located relatively in the 5′-direction and a second primer for amplifying a second target locus which is located in the 3′-direction of the first target locus, a reverse primer of the first primer pair consists of (i) a target hybridization nucleotide sequence that is complementary to a downstream region of the first target locus and (ii) an overlapping sequence that is non-complementary to a target nucleic acid molecule but complementary to a forward primer of the second primer pair. That is, the reverse primer of the first primer pair and the forward primer of the second primer pair may be annealed through the overlapping sequences. Therefore, according to the method of the present invention, mutually independent multiple target loci can be assembled into a single shortened nucleic acid sequence by using the overlapping sequences, and the target loci can be assembled in a substantially accurate order depending on the primer pairs used.

According to the method of the present invention, multiple target loci including preferably at least two target loci, more preferably at least three target loci, still more preferably at least four target loci, still more preferably at least five target loci, and the most preferably at least nine target loci can be assembled into a nucleic acid sequence which is shortened to a single molecule.

According to a preferable embodiment of the present invention, the target nucleic acid molecule may include at least three target loci and the primary amplification primer set used in the step (b) may include at least three primer pairs, the at least three primer pairs including a first primer pair for amplifying a first target locus which is located relatively farthest in the 5′ direction, a second primer pair for amplifying a second target locus which is located in the 3′ direction of the first target locus, and a third primer pair for amplifying a third target locus which is located in the 3′ direction of the second target locus. A reverse primer of the first primer pair may include (i) a target hybridization nucleotide sequence that is complementary to a downstream region of the first target locus and (ii) an overlapping sequence that is non-complementary to the target nucleic acid molecule but complementary to a forward primer of the second primer pair. The forward primer of the second primer pair may include (i) a target hybridization nucleotide sequence that is complementary to an upstream region of the second target locus and (ii) an overlapping sequence that is non-complementary to the target nucleic acid molecule but complementary to the reverse primer of the first primer pair, and a reverse of the second primer pair may include (i) a target hybridization nucleotide sequence that is complementary to a downstream region of the second target locus and (ii) an overlapping sequence that is non-complementary to the target nucleic acid molecule but complementary to a reverse primer of the third primer pair. A forward primer of the third primer pair may include (i) a target hybridization nucleotide sequence that is complementary to an upstream region of the third target locus and (ii) an overlapping sequence that is non-complementary to the target nucleic acid molecule but complementary to the reverse primer of the second primer pair.

According to a preferable embodiment of the present invention, the target nucleic acid molecule may include at least four target loci and the primary amplification primer set used in the step (b) may include at least four primer pairs, the at least four primer pairs including a first primer pair for amplifying a first target locus which is located relatively farthest in the 5′ direction, a second primer pair for amplifying a second target locus which is located in the 3′ direction of the first target locus, a third primer pair for amplifying a third target locus which is located in the 3′ direction of the second target locus, and a fourth primer pair for amplifying a fourth target locus which is located in the 3′ direction of the third target locus. A reverse primer of the first primer pair may include (i) a target hybridization nucleotide sequence that is complementary to a downstream region of the first target locus and (ii) an overlapping sequence that is non-complementary to the target nucleic acid molecule but complementary to a forward primer of the second primer pair. The forward primer of the second primer pair may include (i) a target hybridization nucleotide sequence that is complementary to an upstream region of the second target locus and (ii) an overlapping sequence that is non-complementary to the target nucleic acid molecule but complementary to the reverse primer of the first primer pair, and a reverse of the second primer pair may include (i) a target hybridization nucleotide sequence that is complementary to a downstream region of the second target locus and (ii) an overlapping sequence that is non-complementary to the target nucleic acid molecule but complementary to a reverse primer of the third primer pair. A forward primer of the third primer pair may include (i) a target hybridization nucleotide sequence that is complementary to an upstream region of the third target locus and (ii) an overlapping sequence that is non-complementary to the target nucleic acid molecule but complementary to the reverse primer of the second primer pair. A forward primer of the fourth primer pair may include (i) a target hybridization nucleotide sequence that is complementary to an upstream region of the third target locus and (ii) an overlapping sequence that is non-complementary to the target nucleic acid molecule but complementary to a reverse primer of the third primer pair.

According to a preferable embodiment of the present invention, the target nucleic acid molecule may include at least five target loci and the primary amplification primer set used in the step (b) may include at least four primer pairs, at least four primer pairs including a first primer pair for amplifying a first target locus which is located relatively farthest in the 5′ direction, a second primer pair for amplifying a second target locus which is located in the 3′ direction of the first target locus, a third primer pair for amplifying a third target locus which is located in the 3′ direction of the second target locus, a fourth primer pair for amplifying a fourth target locus which is located in the 3′ direction of the third target locus, and a fifth primer pair for amplifying a fifth target locus which is located in the 3′ direction of the fourth target locus. A reverse primer of the first primer pair may include (i) a target hybridization nucleotide sequence that is complementary to a downstream region of the first target locus and (ii) an overlapping sequence that is non-complementary to the target nucleic acid molecule but complementary to a forward primer of the second primer pair. The forward primer of the second primer pair may include (i) a target hybridization nucleotide sequence that is complementary to an upstream region of the second target locus and (ii) an overlapping sequence that is non-complementary to the target nucleic acid molecule but complementary to the reverse primer of the first primer pair, and a reverse of the second primer pair may include (i) a target hybridization nucleotide sequence that is complementary to a downstream region of the second target locus and (ii) an overlapping sequence that is non-complementary to the target nucleic acid molecule but complementary to a reverse primer of the third primer pair. A forward primer of the third primer pair may include (i) a target hybridization nucleotide sequence that is complementary to an upstream region of the third target locus and (ii) an overlapping sequence that is non-complementary to the target nucleic acid molecule but complementary to the reverse primer of the second primer pair. A forward primer of the fourth primer pair may include (i) a target hybridization nucleotide sequence that is complementary to an upstream region of the fourth target locus and (ii) an overlapping sequence that is non-complementary to the target nucleic acid molecule but complementary to a reverse primer of the third primer pair. A forward primer of the fifth primer pair may include (i) a target hybridization nucleotide sequence that is complementary to an upstream region of the fifth target locus and (ii) an overlapping sequence that is non-complementary to the target nucleic acid molecule but complementary to a reverse primer of the fourth primer pair.

According to a preferable embodiment of the present invention, the primary amplification products in step (b) of the present invention include 70˜150 bp amplicons.

As used herein, the term “complementary” refers to having such complementarity to be selectively hybridizable with the foregoing nucleic acid sequence under specific hybridization or annealing conditions, and refers to encompassing “substantially complementary” and “perfectly complementary”, and preferably, refers to being perfectly complementary.

As used herein, the term “amplification reaction” refers to an amplification reaction of a target nucleic acid molecule. Various amplification reactions have been reported in the art, including a polymerase chain reaction (PCR) (U.S. Pat. Nos. 4,683,195, 4,683,202, and 4,800,159), a reverse transcription-polymerase chain reaction (RT-PCR) (Sambrook et al., Molecular Cloning. A Laboratory Manual, 3rd ed. Cold Spring Harbor Press (2001)), methods of Miller, H. I. (WO 89/06700), and Davey, C. et al. (EP 329,822), multiplex PCR (McPherson and Moller, 2000), a ligase chain reaction (LCR) (17, 18), Gap-LCR (WO 90/01069), a repair chain reaction (EP 439,182), transcription-mediated amplification (TMA) (19) (WO 88/10315), self sustained sequence replication (20) (WO 90/06995), selective amplification of target polynucleotide sequences (U.S. Pat. No. 6,410,276), a consensus sequence primed polymerase chain reaction (CP-PCR) (U.S. Pat. No. 4,437,975), an arbitrarily primed polymerase chain reaction (AP-PCR) (U.S. Pat. Nos. 5,413,909 and 5,861,245), nucleic acid sequence based amplification (NASBA) (U.S. Pat. Nos. 5,130,238, 5,409,818, 5,554,517, and 6,063,603), and strand displacement amplification (21, 22), but are not limited thereto. Other usable amplification methods are described in U.S. Pat. Nos. 5,242,794, 5,494,810, and 4,988,617, and U.S. patent application Ser. No. 09/854,317.

According to the most preferable embodiment of the present invention, the amplification procedure is performed following the PCR disclosed in U.S. Pat. Nos. 4,683,195, 4,683,202, and 4,800,159.

PCR is the most known method of nucleic acid amplification, and its modifications and applications have been developed. For example, in order to improve specificity or sensitivity of PCR, a touchdown PCR, a hot start PCR, a nested PCR, and a booster PCR haven been developed through modification of the conventional PCR procedure. Further, a multiplex PCR, a real-time PCR, a differential display PCR (DD-PCR), rapid amplification of cDNA ends (RACE), an inverse polymerase chain reaction (IPCR), a vectorette PCR, and a thermal asymmetric interlaced PCR (TAIL-PCR) have been developed for specific applications. Detailed descriptions of PCR are shown in McPherson, M. J., and Moller, S. G. PCR. BIOS Scientific Publishers, Springer-Verlag New York Berlin Heidelberg, N.Y. (2000), the teaching of which is incorporated by reference herein.

The target nucleic acid molecule usable herein is not particularly limited, and includes preferably DNA (gDNA or cDNA) and RNA, more preferably DNA, and still more preferably genomic DNA. Further, the target nucleic acid molecule includes, for example, nucleic acids of prokaryotic cells, nucleic acids of eukaryotic cells (for example, protozoa, parasites, fungi, yeasts, higher plants, lower animals, and higher animals including mammals and human beings), nucleic acids of viruses (for example, Herpes virus, HIV, influenza virus, Epstein-Barr virus, hepatitis virus, polio virus, etc.), and viroid nucleic acids.

When the target nucleic acid molecule of the present invention is DNA, multiple target loci can be directly and simultaneously detected through PCR using the primer sets of the present invention.

When RNA is used as a target nucleic acid molecule, the method of the present invention further includes (a-1) obtaining cDNA by reverse-transcription of the target nucleic acid molecule obtained from a sample. The term “sample” used while the assembling method and the detecting method of the present invention are recited includes, but is not limited to, blood, cells, cell materials, tissues, and organs, in which the target nucleic acid molecule of the present invention is included.

In order to obtain RNA as a target nucleic acid molecule, total RNAs are isolated from the sample. The isolation of total RNAs may be performed following the general methods known in the art (see, Sambrook, J. et al., Molecular Cloning. A Laboratory Manual, 3rd ed. Cold Spring Harbor Press (2001); Tesniere, C. et al., Plant Mol. Biol. Rep., 9:242 (1991); Ausubel, F. M. et al., Current Protocols in Molecular Biology, John Willey & Sons (1987); and Chomczynski, P. et al., Anal. Biochem. 162:156 (1987)). For example, total RNAs in the cells may be easily isolated by using Trizol. Then, cDNA is synthesized from the isolated mRNA, and the cDNA is amplified. When total RNAs of the present invention are isolated from the human sample, mRNA has a poly-A tail at a terminal thereof. The oligo dT primer using this sequence characteristic and a reverse transcription enzyme are used to easily synthesize cDNA (see, PNAS USA, 85:8998 (1988); Libert F, et al., Science, 244:569 (1989); and Sambrook, J. et al., Molecular Cloning. A Laboratory Manual, 3rd ed. Cold Spring Harbor Press (2001)). Then, the synthesized cDNA is amplified through a gene amplification reaction.

The primer used herein is hybridized or annealed with one region of the template to form a double-chain structure. The hybridization conditions suitable for forming the double-chain structure are disclosed in Joseph Sambrook, et al., Molecular Cloning, A Laboratory Manual, Cold Spring Harbor Laboratory Press, Cold Spring Harbor, N.Y. (2001), and Haymes, B. D., et al., Nucleic Acid Hybridization, A Practical Approach, IRL Press, Washington, D.C. (1985).

Various DNA polymerases may be used in the amplification of the present invention, and include the “Klenow” fragment of E. coli DNA polymerase I, a thermostable DNA polymerase, and a bacteriophage T7 DNA polymerase. Preferably, the polymerases are thermostable DNA polymerases that may be obtained from various bacterial species, and these include DNA polymerases of Thermus aquaticus (Taq), Thermus thermophilus (Tth), Thermus filiformis, Thermis flavus, Thermococcus literalis, Pyrococcus furiosus (Pfu), Thermus antranikianii, Thermus caldophilus, Thermus chliarophilus, Thermus flavus, Thermus igniterrae, Thermus lacteus, Thermus oshimai, Thermus ruber, Thermus rubens, Thermus scotoductus, Thermus silvanus, Thermus species Z05, Thermus species sps 17, Thermus thermophilus, Thermotoga maritima, Thermotoga neapolitana, and Thermosipho africanus.

When the polymerization reaction is performed, excessive amounts of components necessary for the reaction are preferably provided in a reaction container. The excessive amounts of components necessary for the amplification reaction means such amounts that the amplification reaction is substantially limited by concentrations of the components. Cofactors such as Mg²⁺, and dATP, dCTP, dGTP and dTTP are desirably provided to the reaction mixture such that the degree of amplification can be achieved. All enzymes used in the amplification reaction may be in an active state under the same reaction conditions. In fact, the buffer enables all the enzymes to be close to the optimum reaction conditions. Therefore, the amplification procedure of the present invention may be performed in a single reaction material without changing conditions, such as addition of reaction materials.

The annealing herein is performed under the strict conditions allowing specific combination between target nucleotide sequences and primers. The strict conditions for annealing are sequence-dependent and are various according to the surrounding environmental variables. The thus amplified target gene is a target nucleic acid molecule including multiple target loci on one molecule thereof, and allows the simultaneous analysis of the multiple target loci.

In accordance with another aspect of the present invention, there is provided a method for simultaneously detecting multiple target loci, the method including: (a) obtaining a target nucleic acid molecule including multiple target loci including at least two target loci on a molecule thereof; (b) obtaining primary amplification products by primary amplification of the target nucleic molecule using a primary amplification primer set including at least two primer pairs for being hybridized with upstream and downstream regions of the at least two target loci and amplifying flanking regions of the at least two target loci, wherein the at least two primer pairs each have a forward primer and a reverse primer and the at least two primer pairs include a first primer pair for amplifying a first target locus which is located relatively in the 5′ direction and a second primer pair for amplifying a second target locus which is located in the 3′ direction of the first target locus; wherein a reverse primer of the first primer pair includes (i) a target hybridization nucleotide sequence that is complementary to a downstream region of the first target locus and (ii) an overlapping sequence that is non-complementary to the target nucleic acid molecule but complementary to a forward primer of the second primer pair; and wherein the forward primer of the second primer pair includes (i) a target hybridization nucleotide sequence that is complementary to an upstream region of the second target locus and (ii) an overlapping sequence that is non-complementary to the target nucleic acid molecule but complementary to the reverse primer of the first primer pair; (c) obtaining secondary amplification products by secondary amplification using a secondary amplification primer set and the primary amplification products, the secondary amplification primer set including a primer that is complementary to a 5′ end region formed when the primary amplification products are arranged in the 5′ to 3′ direction and a primer that is complementary to a 3′ end region of the sequence, wherein the secondary amplification products constitute a nucleic acid sequence in which the at least two target loci are located adjacent to each other, the nucleic acid being extended to have a greater length than the target nucleic acid molecule used in step (a); and (d) analyzing the presence or absence of the at least two target loci in the secondary amplification products.
In accordance with still another aspect of the present invention, there is provided a kit for detecting multiple target loci, the kit including the primary amplification primer set and the secondary primer set.

Since the method of the present invention includes the foregoing assembling method, the same descriptions as in the assembling method of the present invention are omitted to avoid excessive complication of the specification due to repetitive descriptions thereof.

According to a preferred embodiment of the present invention, the target loci of the present invention include loci of nucleotide variations.

According to a preferred embodiment of the present invention, the nucleotide variations of the present invention include single nucleotide mutation (point mutation), insertion mutation, and deletion mutation, and more preferably single nucleotide mutation.

The single nucleotide mutation includes single nucleotide polymorphisms (SNPs), frame shift mutation, missense mutation, and nonsense mutation, and the most preferably SNPs.

According to a preferred embodiment of the present invention, the analyzing of the present invention is performed through sequencing.

As used herein, the term “single nucleotide polymorphisms (SNPs)” refers to DNA sequence diversity occurring when a single nucleotide (A, T, C, or G) in the genome differs between members of species or between paired chromosomes of an individual. For example, when there is a single nucleotide difference, like in three DNA fragments from different individuals (see, Table 1, AAGT[A/A]AG, AAGT[A/G]AG, and AAGT[G/G]AG of rs1061147, which are SNP markers of the age-related macular degeneration in the present invention), it is called two alleles (A or G), and almost all SNPs generally have two alleles. In a population, SNPs may be assigned to minor allele frequency (MAF; the lowest allele frequency on the gene locus discovered in the particular population). Variations are present in the human population, and one SNP allele that is common in the geological or ethnic group is very rare. The single nucleotide may be altered (substituted), removed (deleted) or added (inserted) on the polynucleotide sequence. The SNP may induce alterations of translation flames.

In addition, the single nucleotide polymorphisms may be included in coding sequences of genes, non-coding regions of genes, or intergenic regions between genes. SNPs within the coding sequence of a gene may not necessarily cause alterations of an amino acid sequence of the target protein due to the degeneracy of the genetic code. A SNP in which both forms lead to the same polypeptide sequences is termed synonymous (sometimes called a silent mutation)—if a different polypeptide sequences is produced they are non-synonymous. The non-synonymous SNP may be missense or nonsense. While the missense alteration generates a different amino acid, the nonsense alteration forms a non-mature stop codon. The SNP located in a protein-non-coding region may cause gene silencing, transcription factor binding, or the sequence of non-coding RNA.

According to the present invention, the method and kit of the present invention can detect nucleotide variations including the foregoing SNPs very conveniently and effectively. That is, the method and kit of the present invention can provide important approaches and means for realizing a concept of customized drugs, by easily detecting variations (for example, SNPs) on the human DNA sequences that can affect how humans develop diseases and respond to pathogens, chemicals, drugs, vaccines and other agents. Above all things, SNPs, which are actively developed as a marker in recent years, are very important in biomedical researches of diagnosing diseases by comparing genome regions between groups with diseases and groups without diseases. SNP is the major variation of a human genome, and it is speculated that one SNP exists in genome per 1.9 kb (Sachidanandam et al., 2001). SNP is a very stable genetic marker, and occasionally directly influences on the phenotype, and thus is very suitable for automatic genotype-determining system (Landegren et al., 1998; Isaksson et al., 2000). In addition, studies on SNPs are important for cereal crops and livestock cultivation programs.

The features and advantages of the present invention will be summarized as follows:

(a) The present invention is directed to a method for assembling multiple target loci into a single shortened nucleic acid sequence and a method for simultaneously detecting multiple target loci using the method.

(b) The method of the present invention enables multiple target loci to be assembled into a single shortened nucleic acid sequence through two PCRs, that is, primary polymerase chain reaction (PCR) and secondary PCR.

(c) More specifically, each of primary amplification primer pairs (a forward primer and a reverse primer) used herein includes a target-specific sequence (target hybridization nucleotide sequence) and a 5′-flanking assembly spacer sequence (overlapping sequence).

(d) Further, primary amplification products obtained by amplification using the primary amplification primer pairs are conveniently and easily assembled to a single shortened nucleic acid sequence through the secondary amplification primer set, so that multiple target loci can be simultaneously detected.

(e) Therefore, the method and kit of the present invention enable simultaneous detection and analysis of multiple variations (for example, SNPs) of the DNA sequence of (preferably, bloods of the human being), thereby significantly reducing the sequencing cost for detection of variations and providing important approaches and means for realizing a concept of customized drugs.

The present invention will now be described in further detail by examples. It would be obvious to those skilled in the art that these examples are intended to be more concretely illustrative and the scope of the present invention as set forth in the appended claims is not limited to or by the examples.

### EXAMPLES

### Materials and Methods

### Oligonucleotide Sequence Design

We used the computer program Perl-mTAS to generate mTAS oligonucleotides of an optimal length to anneal at specific temperatures. Oligonucleotide probes were constructed from target-specific sequences and 5′-flanking assembly spacer sequences. All were approximately 25 bp and were annealed at Tm 60oC. For each target genomic locus, a 7 bp gap was introduced (i.e., 3 bp for the left, 1 bp for the SNP, 3 bp for the right). Although assembly spacer sequences were randomly generated, annealing regions on the assembly sequences were determined based on nearest neighbor methods (19) to calculate the temperature values for any overlapping regions between oligonucleotides.

mTAS Target Sequencing Using Genomic DNA Purified from Human Blood

We prepared genomic DNA using the AccuPrep™ Genomic DNA Extraction Kit (Bioneer, Korea) with blood samples obtained from healthy volunteers. All oligonucleotides were obtained from commercial vendors (Marcrogen, Korea; Bioneer, Korea). The Oligonucleotide sequences are listed in Tables 1-9.

TABLE 1
List of target loci of oligonucleotides used for mTAS
(SNP detection).1

| | Disease | | Restriction | | | |
|---|---|---|---|---|---|---|
| | or | | Enzyme | | | |
| Set | phenotype | # of | for | | | |
| # | Name | loci | cloning | loci | Primer (5′->3′) | |
| 1 | Age- | 3 | BglII/ | rs1061147 | forward | GGTGGTAGATCTTGCAACCCGGGGAAA |
| | related | | XhoI | | | TAC (SEQ ID NO: 1) |
| | Macular | | | | reverse | CCGTTCGTCGGAAAACATTGCCAGCCA |
| | Degeneration | | | | | GTACTTGTGCA |
| | | | | | | (SEQ ID NO: 2) |
| | | | | rs547154 | forward | CAATGTTTTCCGACGAACGGTTCCCGC |
| | | | | | | CCGCCAGAGGCC |
| | | | | | | (SEQ ID NO: 3) |
| | | | | | reverse | CACCGGGCACAGTTACTGGCACTGTGT |
| | | | | | | CCAGGTTCC (SEQ ID NO: 4) |
| | | | | rs3750847 | forward | CAGTAACTGTGCCCGGTGTAGGACATG |
| | | | | | | ACAAGCTGTCATTCAAGACC |
| | | | | | | (SEQ ID NO: 5) |
| | | | | | reverse | GGTGGTCTCGAGAGCCCCAGGCAGCC |
| | | | | | | (SEQ ID NO: 6) |
| 2 | Alpha-1 | 2 | BglII/ | Rs17580 | forward | GGTGGTAGATCTGATGATATCGTGGGT |
| | Antitrypsin | | XhoI | | | GAGTTCA (SEQ ID NO: 7) |
| | Deficiency | | | | reverse | TCATTGTCTGACTGGCTGACGAGGGGA |
| | | | | | | AACTACAGCACC |
| | | | | | | (SEQ ID NO: 8) |
| | | | | Rs2892947 | forward | GTCAGCCAGTCAGACAATGACTCGCCC |
| | | | | 4 | | ACCACCTTCACTCCCTT |
| | | | | | | (SEQ ID NO: 9) |
| | | | | | reverse | GGTGGTCTCGAGAAGGCTGTGCTGACC |
| | | | | | | ATC (SEQ ID NO: 10) |
| 3 | BRCA | 3 | BglII/ | 185delAG | forward | GGTGGTGGTGGTAGATCTTTGTGCTGA |
| | Cancer | | XhoI | | | CTTACCAGATGG |
| | Mutations | | | | | (SEQ ID NO: 11) |
| | | | | | reverse | ATCGATTCAGATGCTTTCACAACATGT |
| | | | | | | CATTAATGCTATGCAGAAAATCTT |
| | | | | | | (SEQ ID NO: 12) |
| | | | | 5382insC | forward | GTTGTGAAAGCATCTGAATCGATGGAG |
| | | | | | | CTTTACCTTTCTGTCCTG |
| | | | | | | (SEQ ID NO: 13) |
| | | | | | reverse | GTCTCAATCGTCCGAAATCTTAAAAGG |
| | | | | | | TCCAAAGCGAGCAAG |
| | | | | | | (SEQ ID NO: 14) |
| | | | | 6174delT | forward | TTAAGATTTCGGACGATTGAGACCTTG |
| | | | | | | TGGGATTTTTAGCACAGC |
| | | | | | | (SEQ ID NO: 15) |
| | | | | | reverse | GGTGGTGGTGGTCTCGAGCATCTGATA |
| | | | | | | CCTGGACAGATTTTC |
| | | | | | | (SEQ ID NO: 16) |
| 4 | Clopidogrel | 5 | BglII/ | rs4244285 | forward | GGTGGTAGATCTGCAATAATTTTCCCA |
| | (Plavix ®) | | XhoI | | | CTATCATTGATTATTT |
| | Efficacy | | | | | (SEQ ID NO: 17) |
| | | | | | reverse | CTGGGATCGATTAAGTAAGTTGAACGC |
| | | | | | | AAGGTTTTTAAGTAATTTGTTATGGGT |
| | | | | | | (SEQ ID NO: 18) |
| | | | | rs4986893 | forward | GTTCAACTTACTTAATCGATCCCAGAT |
| | | | | | | CAGGATTGTAAGCACCCC |
| | | | | | | (SEQ ID NO: 19) |
| | | | | | reverse | CTACGACCGATCGCAATCAGCAAAAAA |
| | | | | | | CTTGGCCTTACCTG |
| | | | | | | (SEQ ID NO: 20) |
| | | | | rs2839950 | forward | TCATTCCCATCCCTCCTACACCTACCT |
| | | | | 4 | | CTTAACAAGAGGAGAAGGCT |
| | | | | | | (SEQ ID NO: 21) |
| | | | | | reverse | CCTGGCCCTTCAGAGGTATCGCACAAG |
| | | | | | | GACCACAAAAGGAT |
| | | | | | | (SEQ ID NO: 22) |
| | | | | rs4129155 | forward | GATACCTCTGAAGGGCCAGGATCAGGG |
| | | | | 6 | | AATCGTTTTCAGCAATGGAAAG |
| | | | | | | (SEQ ID NO: 23) |
| | | | | | reverse | CGAGCTGATCTGGTGGCAGAAACGCCG |
| | | | | | | GATCTCCT (SEQ ID NO: 24) |
| | | | | rs1224856 | forward | GCCACCAGATCAGCTCGATCAGACGTT |
| | | | | | | CAAATTTGTGTCTTCTGTTCTCA |
| | | | | | | (SEQ ID NO: 25) |
| | | | | | reverse | GGTGGTCTCGAGGGCGCATTATCTCTT |
| | | | | | | ACATCAGA (SEQ ID NO: 26) |

TABLE 2
List of target loci of oligonucleotides used for mTAS
(SNP detection).

| | | | Restric- | | | |
|---|---|---|---|---|---|---|
| | Disease | | tion | | | |
| | or | | Enzyme | | | |
| Set | phenotype | # of | for | | | |
| # | Name | loci | cloning | loci | Primer (5′->3′) | |
| 5 | Celiac | 2 | BglII/ | rs2187668 | forward | GGTGGTAGATCTAACAATCATTTTACC |
| | Disease | | XhoI | | | ACATGGTCC (SEQ ID NO: 27) |
| | | | | | reverse | GGGCGTGGGCTAATGTATTAACCACAT |
| | | | | | | ATGAGGCAGCTGAGAG |
| | | | | | | (SEQ ID NO: 28) |
| | | | | rs6822844 | forward | GTTAATACATTAGCCCACGCCCATATG |
| | | | | | | TCTCGCTCTCCATAGCAA |
| | | | | | | (SEQ ID NO: 29) |
| | | | | | reverse | GGTGGTCTCGAGGTGGCAACATGAAAA |
| | | | | | | GAGTCC (SEQ ID NO: 30) |
| 6 | Hemochromatosis | 2 | BglII/ | rs1800562 | forward | GGTGGTAGATCTCCTGGGGAAGAGCAG |
| | | | XhoI | | | AGATATA (SEQ ID NO: 31) |
| | | | | | reverse | CTACGGATCACTCACTTGTAACTTAGC |
| | | | | | | CTGGGTGCTCCACC |
| | | | | | | (SEQ ID NO: 32) |
| | | | | rs1799945 | forward | TAAGTTACAAGTGAGTGATCCGTAGGA |
| | | | | | | CCAGCTGTTCGTGTTCTAT |
| | | | | | | (SEQ ID NO: 33) |
| | | | | | reverse | GTTTTGCTTCGCACAAAAAGTGTCCAC |
| | | | | | | ACGGCGACTCT |
| | | | | | | (SEQ ID NO: 34) |
| | Parkinson's | 1 | | rs3463758 | forward | CACTTTTTGTGCGAAGCAAAACGATCC |
| | Disease | | | 6 | | ATCATTGCAAAGATTGCTGAC |
| | | | | | | (SEQ ID NO: 35) |
| | | | | | reverse | GGTGGTCTCGAGCATTCTACAGCAGTA |
| | | | | | | CTGAGCAA (SEQ ID NO: 36) |
| 7 | Psoriasis | 3 | BglII/ | rs1048455 | forward | GGTGGTAGATCTAGGTCCCCTTCCTCC |
| | | | XhoI | 4 | | TATCT (SEQ ID NO: 37) |
| | | | | | reverse | CTGGGATCGATTAAGTAAGTTGAACGG |
| | | | | | | CAGGCTGAGACGTC |
| | | | | | | (SEQ ID NO: 38) |
| | | | | rs3212227 | forward | GTTCAACTTACTTAATCGATCCCAG |
| | | | | | | CTGATTGTTTCAATGAGCATTTAGC |
| | | | | | | (SEQ ID NO: 39) |
| | | | | | reverse | CTACGACCGATCGCAATCAGCAAAA |
| | | | | | | TCACAATGATATCTTTGCTGTATTTGT |
| | | | | | | ATA (SEQ ID NO: 40) |
| | | | | rs1120902 | forward | TGATTGCGATCGGTCGTAGAGGTAG |
| | | | | 6 | | TTCTTTGATTGGGATATTTAACAGATC |
| | | | | | | AT (SEQ ID NO: 41) |
| | | | | | reverse | GGTGGTCTCGAGGAAATTCTGCAAAAA |
| | | | | | | CCTACCCA (SEQ ID NO: 42) |
| 8 | Prostate | 5 | EcoRI/ | rs1447295 | forward | GGTGGTGAATTCTGCCATTGGGGAGGT |
| | Cancer | | NotI | | | ATGTA (SEQ ID NO: 43) |
| | | | | | reverse | CAATGTAGAAAGCCAGGGTCTAGGTTC |
| | | | | | | CTGTTGCTTTTTTTCCATAG |
| | | | | | | (SEQ ID NO: 44) |
| | | | | rs6983267 | forward | TAGACCCTGGCTTTCTACATTGCAACC |
| | | | | | | TTTGAGCTCAGCAGATGA |
| | | | | | | (SEQ ID NO: 45) |
| | | | | | reverse | TGACGGGCACTTAGTCCTCGCACATAA |
| | | | | | | AAATTCTTTGTACTTTTCTCA |
| | | | | | | (SEQ ID NO: 46) |
| | | | | rs1050548 | forward | TGACGGGCACTTAGTCCTCGCACATAA |
| | | | | 3 | | AAATTCTTTGTACTTTTCTCA |
| | | | | | | (SEQ ID NO: 47) |
| | | | | | reverse | CCGAGATTAGTTCTGGAACGTCTCTGT |
| | | | | | | TCTAAGGCTCATGGC |
| | | | | | | (SEQ ID NO: 48) |
| | | | | rs1859962 | forward | GACGTTCCAGAACTAATCTCGGAATAC |
| | | | | | | TTTTCCAAATCCCTGCCC |
| | | | | | | (SEQ ID NO: 49) |
| | | | | | reverse | GCGTCAGTGTGCAGATCAAAATCTTGG |
| | | | | | | GACCTTTAAAGTGTTC |
| | | | | | | (SEQ ID NO: 50) |
| | | | | rs4430796 | forward | TGATCTGCACACTGACGCCACGCGGAG |
| | | | | | | AGAGGCAGCACAGACT |
| | | | | | | (SEQ ID NO: 51) |
| | | | | | reverse | GGTGGTGCGGCCGCTGCCCAATTTAAG |
| | | | | | | CTTTATGCAG |
| | | | | | | (SEQ ID NO: 52) |

TABLE 3
List of target loci of oligonucleotides used for mTAS
(SNP detection).

| | Disease | | Restriction | | | |
|---|---|---|---|---|---|---|
| | or | | Enzyme | | | |
| Set | phenotype | # of | for | | | |
| # | Name | loci | cloning | loci | Primer (5′->3′) | |
| 9-1 | Rheumatoid | 3 | EcoRI/ | rs6457617 | forward | GGTGGTGAATTCCCATATGCACAGATC |
| | Arthritis | | XhoI | | | TTTGTTAGTCA (SEQ ID NO: 53) |
| | fragment | | | | reverse | TGCGTCCAGAGAGAACGTATTGTTGAG |
| | 1 | | | | | TCCATGAGCAGAT |
| | | | | | | (SEQ ID NO: 54) |
| | | | | rs1120336 | forward | TACGTTCTCTCTGGACGCATGTTTAGG |
| | | | | 6 | | TCGTGGATATTGCCCAC |
| | | | | | | (SEQ ID NO: 55) |
| | | | | | reverse | CCCATACCGCTGCTCGCTTCTTGGCTG |
| | | | | | | GAGGGC (SEQ ID NO: 56) |
| | | | | rs2476601 | forward | CGAGCAGCGGTATGGGCCGCTTCACCC |
| | | | | | | ACAATAAATGATTCAGGTGTCC |
| | | | | | | (SEQ ID NO: 57) |
| | | | | | reverse | GGTGGTCTCGAGCCCCCTCCACTTCCT |
| | | | | | | GTA (SEQ ID NO: 58) |
| 9-2 | Rheumatoid | 3 | EcoRI/ | rs3890745 | forward | GGTGGTGAATTCCTGAGGGAGGGCCCA |
| | Arthritis | | XhoI | | | A (SEQ ID NO: 59) |
| | fragment | | | | reverse | CGTCCATGCCACTGCGGGGGAAATTGT |
| | 2 | | | | | TACAAATCCAGAC |
| | | | | | | (SEQ ID NO: 60) |
| | | | | rs2327832 | forward | CGCAGTGGCATGGACGAAGATACCGGC |
| | | | | | | ACTTCAATAAAAAAAAATTCTTAAATG |
| | | | | | | AAAAA (SEQ ID NO: 61) |
| | | | | | reverse | GGTAGGCACCTGGCATGACATCTTCAG |
| | | | | | | TTGAGGTGTCCTTT |
| | | | | | | (SEQ ID NO: 62) |
| | | | | rs3761847 | forward | TCATGCCAGGTGCCTACCTTGTGCAGT |
| | | | | | | CCCTTCTCTCCCCTCC |
| | | | | | | (SEQ ID NO: 63) |
| | | | | | reverse | GGTGGTCTCGAGAGAGAGGGTGGTATT |
| | | | | | | GAGGC (SEQ ID NO: 64) |
| 9 | Rheumatoid | 6 | EcoRI/ | rs6457617 | forward | GGTGGTGAATTCCCATATGCACAGATC |
| | Arthritis | | XhoI | | | TTTGTTAGTCA (SEQ ID NO: 65) |
| | long | | | | reverse | TGCGTCCAGAGAGAACGTATTGTTGAG |
| | assemble | | | | | TCCATGAGCAGAT |
| | fragment | | | | | (SEQ ID NO: 66) |
| | | | | rs1120336 | forward | TACGTTCTCTCTGGACGCATGTTTAGG |
| | | | | 6 | | TCGTGGATATTGCCCAC |
| | | | | | | (SEQ ID NO: 67) |
| | | | | | reverse | CCCATACCGCTGCTCGCTTCTTGGCTG |
| | | | | | | GAGGGC (SEQ ID NO: 68) |
| | | | | rs2476601 | forward | CGAGCAGCGGTATGGGCCGCTTCACCC |
| | | | | | | ACAATAAATGATTCAGGTGTCC |
| | | | | | | (SEQ ID NO: 69) |
| | | | | | reverse | CTGGGATCGATTAAGTAAGTTGAACCC |
| | | | | | | CCCTCCACTTCCTGTA |
| | | | | | | (SEQ ID NO: 70) |
| | | | | rs3890745 | forward | GTTCAACTTACTTAATCGATCCCAGCT |
| | | | | | | GAGGGAGGGCCCAA |
| | | | | | | (SEQ ID NO: 71) |
| | | | | | reverse | CGTCCATGCCACTGCGGGGGAAATTGT |
| | | | | | | TACAAATCCAGAC |
| | | | | | | (SEQ ID NO: 72) |
| | | | | rs2327832 | forward | CGCAGTGGCATGGACGAAGATACCGGC |
| | | | | | | ACTTCAATAAAAAAAAATTCTTAAATG |
| | | | | | | AAAAA (SEQ ID NO: 73) |
| | | | | | reverse | GGTAGGCACCTGGCATGACATCTTCAG |
| | | | | | | TTGAGGTGTCCTTT |
| | | | | | | (SEQ ID NO: 74) |
| | | | | rs3761847 | forward | TCATGCCAGGTGCCTACCTTGTGCAGT |
| | | | | | | CCCTTCTCTCCCCTCC |
| | | | | | | (SEQ ID NO: 75) |
| | | | | | Reverse | GGTGGTCTCGAGAGAGAGGGTGGTATT |
| | | | | | | GAGGC (SEQ ID NO: 76) |

TABLE 4
List of target loci of oligonucleotides used for mTAS
(SNP detection).

| | Disease | | Restriction | | | |
|---|---|---|---|---|---|---|
| | or | | Enzyme | | | |
| Set | phenotype | # of | for | | | |
| # | Name | loci | cloning | loci | Primer (5′->3′) | |
| 10- | Type 1 | 4 | EcoRI/ | rs3129934 | forward | GGTGGTGAATTCTCACTCTCGTTATTC |
| 1 | Diabetes | | XhoI | | | TAGGATACATTATATT |
| | fragment | | | | | (SEQ ID NO: 77) |
| | 1 | | | | reverse | CGCTAGCTTTACCGCTCTTCCTTAGTG |
| | | | | | | AAGTGGCCGG (SEQ ID NO: 78) |
| | | | | rs3087243 | forward | AAGAGCGGTAAAGCTAGCGGACTGCTG |
| | | | | | | ATTTCTTCACCACTATTTGGGATAT |
| | | | | | | (SEQ ID NO: 79) |
| | | | | | reverse | TCAACCTCATATGGTAATCGGGAGGAC |
| | | | | | | TGCTATGTCTGTGTTAAC |
| | | | | | | (SEQ ID NO: 80) |
| | | | | rs1990760 | forward | CCCGATTACCATATGAGGTTGATCGTC |
| | | | | | | GGCACACTTCTTTTGCA |
| | | | | | | (SEQ ID NO: 81) |
| | | | | | reverse | GAAGTTATGAAGGGTCATTCTGCAGGG |
| | | | | | | AACTTTACATTGTAAGAGAAAAC |
| | | | | | | (SEQ ID NO: 82) |
| | | | | rs3741208 | forward | GCAGAATGACCCTTCATAACTTCATCG |
| | | | | | | GTTGTTGCCTCTCCC |
| | | | | | | (SEQ ID NO: 83) |
| | | | | | reverse | GGTGGTCTCGAGTGGACAGGAGACTGA |
| | | | | | | GGAG (SEQ ID NO: 84) |
| 10- | Type 1 | 4 | EcoRI/ | rs1893217 | forward | GGTGGTGAATTCCACTTGTCACCATTC |
| 2 | Diabetes | | XhoI | | | CTAGGG (SEQ ID NO: 85) |
| | fragment | | | | reverse | CCGATGCGCTGGACTATTAGATACACT |
| | 2 | | | | | CTTCTTCCTCTACCT |
| | | | | | | (SEQ ID NO: 86) |
| | | | | rs2476601 | forward | AATAGTCCAGCGCATCGGAATGCGTCC |
| | | | | | | ACAATAAATGATTCAGGTGTCC |
| | | | | | | (SEQ ID NO: 87) |
| | | | | | reverse | TTTGCCTAACTTGCGCATTTCCCCCTC |
| | | | | | | CACTTCCTGTA (SEQ ID NO: 88) |
| | | | | rs3184504 | forward | AAATGCGCAAGTTAGGCAAACGCTAGC |
| | | | | | | ATCCAGGAGGTCCGG |
| | | | | | | (SEQ ID NO: 89) |
| | | | | | reverse | CGTACTCAAATCTTACCACGGTTCAAG |
| | | | | | | CCGTGTGCACC (SEQ ID NO: 90) |
| | | | | rs725613 | forward | ACCGTGGTAAGATTTGAGTACGTTCGC |
| | | | | | | TGCCTATCAGTGTTTAGCAC |
| | | | | | | (SEQ ID NO: 91) |
| | | | | | reverse | GGTGGTCTCGAGATCAAGACGCCAGGC |
| | | | | | | AC (SEQ ID NO: 92) |

TABLE 5
List of target loci of oligonucleotides used for mTAS
(SNP detection).

| | Disease | | Restriction | | | |
|---|---|---|---|---|---|---|
| | or | | Enzyme | | | |
| Set | phenotype | # of | for | | | |
| # | Name | loci | cloning | loci | Primer (5′->3′) | |
| 10 | Type 1 | 8 | EcoRI/ | rs3129934 | forward | GGTGGTGAATTCTCACTCTCGTTATTC |
| | Diabetes | | XhoI | | | TAGGATACATTATATT |
| | long | | | | | (SEQ ID NO: 93) |
| | assemble | | | | reverse | CGCTAGCTTTACCGCTCTTCCTTAGTG |
| | fragment | | | | | AAGTGGCCGG (SEQ ID NO: 94) |
| | | | | rs3087243 | forward | AAGAGCGGTAAAGCTAGCGGACTGCTG |
| | | | | | | ATTTCTTCACCACTATTTGGGATAT |
| | | | | | | (SEQ ID NO: 95) |
| | | | | | reverse | TCAACCTCATATGGTAATCGGGAGGAC |
| | | | | | | TGCTATGTCTGTGTTAAC |
| | | | | | | (SEQ ID NO: 96) |
| | | | | rs1990760 | forward | CCCGATTACCATATGAGGTTGATCGTC |
| | | | | | | GGCACACTTCTTTTGCA |
| | | | | | | (SEQ ID NO: 97) |
| | | | | | reverse | GAAGTTATGAAGGGTCATTCTGCAGGG |
| | | | | | | AACTTTACATTGTAAGAGAAAAC |
| | | | | | | (SEQ ID NO: 98) |
| | | | | rs3741208 | forward | GCAGAATGACCCTTCATAACTTCATCG |
| | | | | | | GTTGTTGCCTCTCCC |
| | | | | | | (SEQ ID NO: 99) |
| | | | | | reverse | CTGGGATCGATTAAGTAAGTTGAACTG |
| | | | | | | GACAGGAGACTGAGGAG |
| | | | | | | (SEQ ID NO: 100) |
| | | | | rs1893217 | forward | GTTCAACTTACTTAATCGATCCCAGTG |
| | | | | | | TCACCATTCCTAGGGACA |
| | | | | | | (SEQ ID NO: 101) |
| | | | | | reverse | CCGATGCGCTGGACTATTAGATACACT |
| | | | | | | CTTCTTCCTCTACCT |
| | | | | | | (SEQ ID NO: 102) |
| | | | | rs2476601 | forward | AATAGTCCAGCGCATCGGAATGCGTCC |
| | | | | | | ACAATAAATGATTCAGGTGTCC |
| | | | | | | (SEQ ID NO: 103) |
| | | | | | reverse | TTTGCCTAACTTGCGCATTTCCCCCTC |
| | | | | | | CACTTCCTGTA |
| | | | | | | (SEQ ID NO: 104) |
| | | | | rs3184504 | forward | AAATGCGCAAGTTAGGCAAACGCTAGC |
| | | | | | | ATCCAGGAGGTCCGG |
| | | | | | | (SEQ ID NO: 105) |
| | | | | | reverse | CGTACTCAAATCTTACCACGGTTCAAG |
| | | | | | | CCGTGTGCACC |
| | | | | | | (SEQ ID NO: 106) |
| | | | | rs725613 | forward | ACCGTGGTAAGATTTGAGTACGTTCGC |
| | | | | | | TGCCTATCAGTGTTTAGCAC |
| | | | | | | (SEQ ID NO: 107) |
| | | | | | reverse | GGTGGTCTCGAGATCAAGACGCCAGGC |
| | | | | | | AC (SEQ ID NO: 108) |
| 11- | Type 2 | 5 | EcoRI/ | rs7903146 | forward | GGTGGTGAATTCCAATTAGAGAGCTAA |
| 1 | Diabetes | | XhoI | | | GCACTTTTTAGATA |
| | fragment | | | | | (SEQ ID NO: 109) |
| | 1 | | | | reverse | TCACCTAGGATTAACCATCCCTGTGCC |
| | | | | | | TCATACGGCAATTAAATTATATA |
| | | | | | | (SEQ ID NO: 110) |
| | | | | rs1801282 | forward | AGGGATGGTTAATCCTAGGTGACAACT |
| | | | | | | CTGGGAGATTCTCCTATTGAC |
| | | | | | | (SEQ ID NO: 111) |
| | | | | | reverse | GCTCTGGAACTAAATCTGGACATCAGT |
| | | | | | | GAAGGAATCGCTTTCTG |
| | | | | | | (SEQ ID NO: 112) |
| | | | | rs5219 | forward | TGTCCAGATTTAGTTCCAGAGCGGAGC |
| | | | | | | ACGGTACCTGGGCT |
| | | | | | | (SEQ ID NO: 113) |
| | | | | | reverse | ACGCTGGCCACCAATATTGGCAGAGGA |
| | | | | | | CCCTGCC (SEQ ID NO: 114) |
| | | | | rs4402960 | forward | AATATTGGTGGCCAGCGTTCAAATTAG |
| | | | | | | TAAGGTAGGATGGACAGTAGATT |
| | | | | | | (SEQ ID NO: 115) |
| | | | | | reverse | ACGGATGCAAAGTTGACGAATGTTTGC |
| | | | | | | AAACACAATCAGTATCTT |
| | | | | | | (SEQ ID NO: 116) |
| | | | | rs1111875 | forward | TTCGTCAACTTTGCATCCGTTCATAGA |
| | | | | | | GTGCAGGTTCAGACGTC |
| | | | | | | (SEQ ID NO: 117) |
| | | | | | reverse | GGTGGTCTCGAGCGTACCATCAAGTCA |
| | | | | | | TTTCCTCT (SEQ ID NO: 118) |

TABLE 6
List of target loci of oligonucleotides used for mTAS
(SNP detection).

| | Disease | | Restriction | | | |
|---|---|---|---|---|---|---|
| | or | | Enzyme | | | |
| Set | phenotype | # of | for | | | |
| # | Name | loci | cloning | Loci | Primer (5′->3′) | |
| 11- | Type 2 | 4 | EcoRI/ | rs4712523 | forward | GGTGGTGAATTCTTCTCCTTCTGTTGC |
| 2 | Diabetes | | XhoI | | | ACCC (SEQ ID NO: 119) |
| | fragment | | | | reverse | TGCACGGGATATCATCACGTGTAAATC |
| | 2 | | | | | TTTACATTTGGGTATAAAGGAT |
| | | | | | | (SEQ ID NO: 120) |
| | | | | rs1326663 | forward | CGTGATGATATCCCGTGCACTGATGCT |
| | | | | 4 | | TTATCAACAGCAGCCAGC |
| | | | | | | (SEQ ID NO: 121) |
| | | | | | reverse | AGGTGTTTTAGTTTACTGCTTGTTCGA |
| | | | | | | ACCACTTGGCTGTCCC |
| | | | | | | (SEQ ID NO: 122) |
| | | | | rs1001294 | forward | GAACAAGCAGTAAACTAAAACACCTTG |
| | | | | 6 | | GCTCAAGTGCTCACTCA |
| | | | | | | (SEQ ID NO: 123) |
| | | | | | reverse | CCGATAAGGAGGCTCGAATGGCAGAAT |
| | | | | | | ACCCTCTGGTTTATTCA |
| | | | | | | (SEQ ID NO: 124) |
| | | | | rs2383208 | forward | CATTCGAGCCTCCTTATCGGAGAAACT |
| | | | | | | GTGACAGGAAGGAAGTCC |
| | | | | | | (SEQ ID NO: 125) |
| | | | | | reverse | GGTGGTCTCGAGTTGAAACTAGTAGAT |
| | | | | | | GCTCAATTCATG |
| | | | | | | (SEQ ID NO: 126) |
| 11 | Type 2 | 9 | EcoRI/ | rs7903146 | forward | GGTGGTGAATTCCAATTAGAGAGCTAA |
| | Diabetes | | XhoI | | | GCACTTTTTAGATA |
| | long | | | | | (SEQ ID NO: 127) |
| | assemble | | | | reverse | TCACCTAGGATTAACCATCCCTGTGCC |
| | fragment | | | | | TCATACGGCAATTAAATTATATA |
| | | | | | | (SEQ ID NO: 128) |
| | | | | rs1801282 | forward | AGGGATGGTTAATCCTAGGTGACAACT |
| | | | | | | CTGGGAGATTCTCCTATTGAC |
| | | | | | | (SEQ ID NO: 129) |
| | | | | | reverse | GCTCTGGAACTAAATCTGGACATCAGT |
| | | | | | | GAAGGAATCGCTTTCTG |
| | | | | | | (SEQ ID NO: 130) |
| | | | | rs5219 | forward | TGTCCAGATTTAGTTCCAGAGCGGAGC |
| | | | | | | ACGGTACCTGGGCT |
| | | | | | | (SEQ ID NO: 131) |
| | | | | | reverse | ACGCTGGCCACCAATATTGGCAGAGGA |
| | | | | | | CCCTGCC (SEQ ID NO: 132) |
| | | | | rs4402960 | forward | AATATTGGTGGCCAGCGTTCAAATTAG |
| | | | | | | TAAGGTAGGATGGACAGTAGATT |
| | | | | | | (SEQ ID NO: 133) |
| | | | | | reverse | ACGGATGCAAAGTTGACGAATGTTTGC |
| | | | | | | AAACACAATCAGTATCTT |
| | | | | | | (SEQ ID NO: 134) |
| | | | | rs111875 | forward | TTCGTCAACTTTGCATCCGTTCATAGA |
| | | | | | | GTGCAGGTTCAGACGTC |
| | | | | | | (SEQ ID NO: 135) |
| | | | | | reverse | CTGGGATCGATTAAGTAAGTTGAACCG |
| | | | | | | TACCATCAAGTCATTTCCTCT |
| | | | | | | (SEQ ID NO: 136) |
| | | | | rs4712523 | forward | GTTCAACTTACTTAATCGATCCCAGTT |
| | | | | | | CTCCTTCTGTTGCACCC |
| | | | | | | (SEQ ID NO: 137) |
| | | | | | reverse | TGCACGGGATATCATCACGTGTAAATC |
| | | | | | | TTTACATTTGGGTATAAAGGAT |
| | | | | | | (SEQ ID NO: 138) |
| | | | | rs1326663 | forward | CGTGATGATATCCCGTGCACTGATGCT |
| | | | | 4 | | TTATCAACAGCAGCCAGC |
| | | | | | | (SEQ ID NO: 139) |
| | | | | | reverse | AGGTGTTTTAGTTTACTGCTTGTTCGA |
| | | | | | | ACCACTTGGCTGTCCC |
| | | | | | | (SEQ ID NO: 140) |
| | | | | rs1001294 | forward | GAACAAGCAGTAAACTAAAACACCTTG |
| | | | | 6 | | GCTCAAGTGCTCACTCA |
| | | | | | | (SEQ ID NO: 141) |
| | | | | | reverse | CCGATAAGGAGGCTCGAATGGCAGAAT |
| | | | | | | ACCCTCTGGTTTATTCA |
| | | | | | | (SEQ ID NO: 142) |
| | | | | rs2383208 | forward | CATTCGAGCCTCCTTATCGGAGAAACT |
| | | | | | | GTGACAGGAAGGAAGTCC |
| | | | | | | (SEQ ID NO: 143) |
| | | | | | reverse | GGTGGTCTCGAGTTGAAACTAGTAGAT |
| | | | | | | GCTCAATTCATG |
| | | | | | | (SEQ ID NO: 144) |

TABLE 7
List of target loci of oligonucleotides used for mTAS
(SNP detection).

| | Disease | | Restriction | | | |
|---|---|---|---|---|---|---|
| | or | | Enzyme | | | |
| Set | phenotype | # of | for | | | |
| # | Name | loci | cloning | Loci | Primer (5′->3′) | |
| 12 | Venous | 1 | BglII/ | rs6025 | forward | GGTGGTAGATCTTCAAGGACAAAATAC |
| | Thrombo- | | XhoI | | | CTGTATTCCT |
| | embolism | | | | | (SEQ ID NO: 145) |
| | | | | | reverse | ACGGGTTCAAATGTGGGTATAAGCAGA |
| | | | | | | TCCCTGGACAGGC |
| | | | | | | (SEQ ID NO: 146) |
| | Bipolar | 1 | | rs4948418 | forward | TTATACCCACATTTGAACCCGTTCGCC |
| | Disorder | | | | | TCTGGCATGACAGGGAA |
| | | | | | | (SEQ ID NO: 147) |
| | | | | | reverse | TTCCGCTGAGACTTGACTTTATTTGCT |
| | | | | | | GACTTACCTCAGCC |
| | | | | | | (SEQ ID NO: 148) |
| | Heart | 1 | | rs2383207 | forward | ATAAAGTCAAGTCTCAGCGGAAGCCAC |
| | Attack | | | | | TCCTGTTCGGATCCCTTC |
| | | | | | | (SEQ ID NO: 149) |
| | | | | | reverse | GGTGGTCTCGAGGCTGAAAATAGTAAA |
| | | | | | | TAATCATGCTTAGC |
| | | | | | | (SEQ ID NO: 150) |
| 13 | Colorectal | 3 | EcoRI/ | rs6983267 | forward | GGTGGTGAATTCCTTTGAGCTCAGCAG |
| | Cancer | | NotI | | | ATGAAAG (SEQ ID NO: 151) |
| | | | | | reverse | CTAGAGCCAGTATGTCTCATGCACATA |
| | | | | | | AAAATTCTTTGTACTTTTCTCAGTG |
| | | | | | | (SEQ ID NO: 152) |
| | | | | rs4939827 | forward | GCATGAGACATACTGGCTCTAGCACAG |
| | | | | | | CCTCATCCAAAAGAGGAAA |
| | | | | | | (SEQ ID NO: 153) |
| | | | | | reverse | CATGAGAAGTAGGTCTCACACGGGGAG |
| | | | | | | CTCTGGGGTCCT |
| | | | | | | (SEQ ID NO: 154) |
| | | | | rs3802842 | forward | CGTGTGAGACCTACTTCTCATGCGTCC |
| | | | | | | TTGCAGACCCATAGAAAATCT |
| | | | | | | (SEQ ID NO: 155) |
| | | | | | reverse | GGTGGTGCGGCCGCCCTAAAATGAGGT |
| | | | | | | GAATTTCTGGGA |
| | | | | | | (SEQ ID NO: 156) |
| 14 | Exfolia- | 1 | EcoRI/ | Rs2165241 | forward | GGTGGTGAATTCCTGAGCTCTCAAATG |
| | tion | | NotI | | | CCACA (SEQ ID NO: 157) |
| | Glaucoma | | | | reverse | CCTGTCCCACACCACCTACCCAGGCAT |
| | | | | | | GCCTCTG (SEQ ID NO: 158) |
| | Breast | 2 | | Rs1219648 | forward | TAGGTGGTGTGGGACAGGACGTTCGAG |
| | Cancer | | | | | CACGCCTATTTTACTTGACA |
| | | | | | | (SEQ ID NO: 159) |
| | | | | | reverse | GCTGTAGAAAACCGAAGGATACGGCCA |
| | | | | | | TGGCCATCCTTGAA |
| | | | | | | (SEQ ID NO: 160) |
| | | | | Rs3803662 | forward | CGTATCCTTCGGTTTTCTACAGCTCAG |
| | | | | | | TCCACAGTTTTATTCTTCGCT |
| | | | | | | (SEQ ID NO: 161) |
| | | | | | reverse | CCTGTACGGTTCTTATCCGAGTATCTC |
| | | | | | | TCCTTAATGCCTCTATAGCT |
| | | | | | | (SEQ ID NO: 162) |
| | Lung | 1 | | Rs8034191 | forward | TACTCGGATAAGAACCGTACAGGACAG |
| | Cancer | | | | | CCCAATGTGGTATAAGTTTTCT |
| | | | | | | (SEQ ID NO: 163) |
| | | | | | reverse | GGTGGTGCGGCCGCAGTTACTATCTGT |
| | | | | | | CAGGGCCTT (SEQ ID NO: 164) |
| 15 | Lupus | 4 | BglII/ | Rs9888739 | forward | GGTGGTAGATCTAGTATGCAGAACTCA |
| | (Systemic | | XhoI | | | CTATGTTGTAA |
| | Lupus | | | | | (SEQ ID NO: 165) |
| | Erythe- | | | | reverse | GGCACGGTGATGTGGCAGTCAAAGAGG |
| | matosus) | | | | | TTCTATATTTTTATCATTACAG |
| | | | | | | (SEQ ID NO: 166) |
| | | | | Rs7574865 | forward | GCCACATCACCGTGCCCGCGGATCTAA |
| | | | | | | GTATGAAAAGTTGGTGACCAAAA |
| | | | | | | (SEQ ID NO: 167) |
| | | | | | reverse | GACAGTAGCCATCTTCCAGGGAAATTC |
| | | | | | | CACTGAAATAAGATAACCACT |
| | | | | | | (SEQ ID NO: 168) |
| | | | | Rs2187668 | forward | CCTGGAAGATGGCTACTGTCTCGTGAA |
| | | | | | | CAATCATTTTACCACATGGTCC |
| | | | | | | (SEQ ID NO: 169) |
| | | | | | reverse | GATTTCCCTCAGTTGTGTAGACACACA |
| | | | | | | TATGAGGCAGCTGAGAG |
| | | | | | | (SEQ ID NO: 170) |
| | | | | Rs1048863 | forward | TGTCTACACAACTGAGGGAAATCAAGG |
| | | | | 1 | | CTGCTTCCATAGCTAGTCT |
| | | | | | | (SEQ ID NO: 171) |
| | | | | | reverse | GGTGGTCTCGAGGCCTTGTAGCTCGGA |
| | | | | | | AATGG (SEQ ID NO: 172) |

TABLE 8
List of target loci of oligonucleotides used for mTAS
(SNP detection).

| | Disease | | Restriction | | | |
|---|---|---|---|---|---|---|
| | or | | Enzyme | | | |
| Set | phenotype | # of | for | | | |
| # | Name | loci | cloning | Loci | Primer (5′->3′) | |
| 16 | Multiple | 2 | EcoRI/ | rs6897932 | forward | GGTGGTGAATTCAGGGGAGATGGATCC |
| | Sclerosis | | XhoI | | | TATCTTAC (SEQ ID NO: 173) |
| | | | | | Reverse | AATGGGCCATTCGGCTCGACAGAGAAA |
| | | | | | | AAACTCAAAATGCTG |
| | | | | | | (SEQ ID NO: 174) |
| | | | | rs3135388 | forward | GAGCCGAATGGCCCATTGGGTAATCGT |
| | | | | | | CCTCATCAGGAAAACCTAAAGT |
| | | | | | | (SEQ ID NO: 175) |
| | | | | | reverse | GACTGTACTTTAGGGTAAGCAGATTCA |
| | | | | | | GTAGAGATCTCCCAACAAAC |
| | | | | | | (SEQ ID NO: 176) |
| | Obesity | 1 | | rs3751812 | forward | ATCTGCTTACCCTAAAGTACAGTCCAC |
| | | | | | | CTGAAAATAGGTGAGCTGTC |
| | | | | | | (SEQ ID NO: 177) |
| | | | | | reverse | GGTGGTCTCGAGGAGCCTCTCCCTGCC |
| | | | | | | A (SEQ ID NO: 178) |
| 17 | Ulcerative | 4 | BglII/ | rs2395185 | forward | GGTGGTAGATCTACTACTACACTACAT |
| | Colitis | | XhoI | | | GAAGCCAAAAA |
| | | | | | | (SEQ ID NO: 179) |
| | | | | | reverse | CTGGGATCGATTAAGTAAGTTGAAC |
| | | | | | | ACAGCAGAATTCTCCAGGGA |
| | | | | | | (SEQ ID NO: 180) |
| | | | | rs9858542 | forward | GTTCAACTTACTTAATCGATCCCAG |
| | | | | | | CGAGCAAGCTGGCAAACT |
| | | | | | | (SEQ ID NO: 181) |
| | | | | | reverse | CTACGACCGATCGCAATCAGCAAAA |
| | | | | | | TGCAGGCAGTGCATACC |
| | | | | | | (SEQ ID NO: 182) |
| | | | | rs1088336 | forward | TGATTGCGATCGGTCGTAGAGGTAG |
| | | | | 5 | | TTCGTTCTCAGACGGTTTGAA |
| | | | | | | (SEQ ID NO: 183) |
| | | | | | reverse | CCTGGCCCTTCAGAGGTATCGCACA |
| | | | | | | GGGGTCACGTTGGCAC |
| | | | | | | (SEQ ID NO: 184) |
| | | | | rs1120902 | forward | GATACCTCTGAAGGGCCAGGATCAGTT |
| | | | | 6 | | CTTTGATTGGGATATTTAACAGATCAT |
| | | | | | | CAT (SEQ ID NO: 185) |
| | | | | | reverse | GGTGGTCTCGAGGAAATTCTGCAAAAA |
| | | | | | | CCTACCCA (SEQ ID NO: 186) |
| 18 | Alcohol | 1 | BglII/ | rs671 | forward | GGTGGTAGATCTCGGGCTGCAGGCATA |
| | flush | | XhoI | | | C (SEQ ID NO: 187) |
| | reaction | | | | reverse | CTGTTTTGCGCTCGCGGTCCCACACTC |
| | | | | | | ACAGTTTTCA |
| | | | | | | (SEQ ID NO: 188) |
| | Bitter | 1 | | rs713598 | forward | CGCGAGCGCAAAACAGCGCTTGGACGC |
| | Taste | | | | | ACACAATCACTGTTGCTCA |
| | Perception | | | | | (SEQ ID NO: 189) |
| | | | | | reverse | CCAATGGAAAAGCTGCAGGAGAATTTT |
| | | | | | | TGGGATGTAGTGAAGAGG |
| | | | | | | (SEQ ID NO: 190) |
| | Earwax | 1 | | rs1782293 | forward | TCCTGCAGCTTTTCCATTGGCTAGCAC |
| | Type | | | 1 | | CAAGTCTGCCACTTACTG |
| | | | | | | (SEQ ID NO: 191) |
| | | | | | reverse | GGTGGTCTCGAGGCTTCTGCATTGCCA |
| | | | | | | GTGTA (SEQ ID NO: 192) |
| 19 | Eye Color | 1 | BglII/ | rs1291383 | forward | GGTGGTAGATCTGGCCAGTTTCATTTG |
| | | | XhoI | 2 | | AGCATTAA (SEQ ID NO: 193) |
| | | | | | reverse | AGAGGTAATTCCTTGTGTGCATTAGCG |
| | | | | | | TGCAGAACTTGACA |
| | | | | | | (SEQ ID NO: 194) |
| | Lactose | 1 | | rs4988235 | forward | AATGCACACAAGGAATTACCTCTTCGT |
| | Intolerance | | | | | TCCTTTGAGGCCAGGG |
| | | | | | | (SEQ ID NO: 195) |
| | | | | | reverse | CGCCGGACAAAAGTACTCTGCTGGCAA |
| | | | | | | TACAGATAAGATAATGTAG |
| | | | | | | (SEQ ID NO: 196) |
| | Malaria | 1 | | rs2814778 | forward | AGAGTACTTTTGTCCGGCGGCGTCACC |
| | Resistance | | | | | CTCATTAGTCCTTGGCTCTTA |
| | (Duffy | | | | | (SEQ ID NO: 197) |
| | Antigen) | | | | reverse | TGCCTAACCTCCTTAATCGGATGCGCC |
| | | | | | | TGTGCTTCCAAG |
| | | | | | | (SEQ ID NO: 198) |
| | Muscle | 1 | | rs1815739 | forward | ATCCGATTAAGGAGGTTAGGCAGGCAC |
| | Performance | | | | | TGCCCGAGGCTGAC |
| | | | | | | (SEQ ID NO: 199) |
| | | | | | reverse | GGTGGTCTCGAGGATGGCACCTCGCTC |
| | | | | | | TC (SEQ ID NO: 200) |
| 20 | Norovirus | 1 | BglII/ | rs601338 | forward | GGTGGTAGATCTCCGGCTACCCCTGCT |
| | Resistance | | XhoI | | | (SEQ ID NO: 201) |
| | | | | | reverse | GCCCATATTCCAGGGCCCGCGGAGGTG |
| | | | | | | GTGGTAGAAG (SEQ ID NO: 202) |
| | Restless | 1 | | rs3923809 | forward | GGGCCCTGGAATATGGGCAACATGCAG |
| | Legs | | | | | TGAAAATAAAATGATAGCTTTCTCTCT |
| | Syndrome | | | | | (SEQ ID NO: 203) |
| | | | | | reverse | GGTGGTCTCGAGGTCCTACTGAATTGC |
| | | | | | | AGATGGAT (SEQ ID NO: 204) |

TABLE 9
List of target loci of oligonucleotides used for mTAS
(SNP detection).

| | No. of | | | |
|---|---|---|---|---|
| | target | Mutant | | |
| target | mutant | site | Primer (5′->3′) | |
| EGFR | 3 | Exon 18 | forward | ATCTCGATCCCGCGAAATTAATACGAGATCTGTGGAGAAGCT |
| mutation | | (not | | CCCAACCA (SEQ ID NO: 205) |
| | | targeted) | reverse | CTACGACCGATCGCAATCAGCAAAACTTATACACCGTGCCGA |
| | | | | AC (SEQ ID NO: 206) |
| | | Exon 19 | forward | TGATTGCGATCGGTCGTAGAGGTAGAGGTAAAAGTTAAAATT |
| | | (deletion | | CCCGTCGCTATC (SEQ ID NO: 207) |
| | | mutation) | reverse | CTGGGATCGATTAAGTAAGTTGAACCCTTGTTGGCTTTCGGA |
| | | | | GA (SEQ ID NO: 208) |
| | | Exon 21 | forward | GTTCAACTTACTTAATCGATCCCAGGCATGTCAAGATCACAG |
| | | (Leu858Arg) | | ATTTTGG (SEQ ID NO: 209) |
| | | | reverse | CCTGGCCCTTCAGAGGTATCTGCATGGTATTCTTTCTCTTCC |
| | | | | GCA (SEQ ID NO: 210) |
| | | Exon 20 | forward | GATACCTCTGAAGGGCCAGGCATCTGCCTCACCTCCACC |
| | | (Thr790Met) | | (SEQ ID NO: 211) |
| | | | reverse | GCACGATGCCGGTGAACGCGGCCGCCACCAGTTGAGCAGGTA |
| | | | | CTG (SEQ ID NO: 212) |

* The sequencing primer for detecting EGFR mutation (5′->3′): forward primer, ATCTCGATCCCGCGAAATTAATACG (SEQ ID NO: 213); reverse primer, GCACGATGCCGGTGAAC (SEQ ID NO: 214).

We carried out assembly PCR using these probes to generate multiple amplicons and then used the overlapping spacer sequences in a second-round assembly process to construct the desired long DNA sequence. For the first assembly PCR step, we used mTAS oligonucleotides, genomic DNA, water and h-Taq Premix™ DNA polymerase (Solgent, Korea) to amplify the target DNA sequences. The PCR reaction was initiated by heating at 95° C. for 3 min followed by 40 cycles of the following program: 95° C. for 30 s, 60° C. for 60 s, and 72° C. for 30 s. A final elongation at 72° C. was carried out for 10 min, and the products were stored at 4° C. Using outside flanking primers, 5 μl of the first PCR products, 10 μlh-Taq Premix™ DNA polymerase, and 5 μl of water, we performed the second PCR reaction under the same PCR conditions used in the first PCR reaction. The PCR reaction volume was 20 μl. Refer tables 10 and 11 for details of the PCR reaction conditions).

TABLE 10
First-step Assembly PCR protocol for mTAS.

| | | | Genomic | | |
|---|---|---|---|---|---|
| | | | DNA | | |
| | | | from human | | Expected |
| | 2x taq | | blood | For & Rev | target |
| Sample | premix | Water | (μl, | primer mix | amplicon |
| No. | (μl) | (μl) | 4.3 ng/μl) | (μl, 10 μM) | size (bp) |
| 1 set | 10 | 4 | 1.2 | 6 | 199 |
| 2 set | 10 | 6 | 1.2 | 4 | 141 |
| 3 set | 10 | 4 | 1.2 | 6 | 226 |
| 4 set | 10 | 0 | 1.2 | 10 | 382 |
| 5 set | 10 | 6 | 1.2 | 4 | 149 |
| 6 set | 10 | 4 | 1.2 | 6 | 216 |
| 7 set | 10 | 4 | 1.2 | 6 | 238 |
| 8 set | 10 | 0 | 1.2 | 10 | 379 |
| 9-1 set | 10 | 4 | 1.2 | 6 | 190 |
| 9-2 set | 10 | 4 | 1.2 | 6 | 200 |
| 9 long set | 10 | 4 | 3 | 6 | 406 |
| 10-1 set | 10 | 2 | 1.2 | 8 | 308 |
| 10-2 set | 10 | 2 | 1.2 | 8 | 249 |
| 10 long set | 12 | 4 | 3 | 8 | 564 |
| 11-1 set | 10 | 0 | 1.2 | 10 | 343 |
| 11-2 set | 10 | 2 | 1.2 | 4 | 268 |
| 11 long set | 13 | 4 | 3 | 9 | 624 |
| 12 set | 10 | 4 | 1.2 | 6 | 195 |
| 13 set | 10 | 4 | 1.2 | 6 | 205 |
| 14 set | 10 | 2 | 1.2 | 8 | 298 |
| 15 set | 10 | 2 | 1.2 | 8 | 325 |
| 16 set | 10 | 4 | 1.2 | 6 | 228 |
| 17 set | 10 | 2 | 1.2 | 8 | 297 |
| 18 set | 10 | 4 | 1.2 | 6 | 218 |
| 19 set | 10 | 2 | 1.2 | 8 | 250 |
| 20 set | 10 | 6 | 1.2 | 4 | 149 |

TABLE 11
Second-step Assembly PCR protocol for mTAS.

| 2x taq | | First PCR | First forward | Last reverse |
|---|---|---|---|---|
| premix | Water | amplicon | primer | primer |
| (μl) | (μl) | (μl) | (μl, 10 μM) | (μl, 10 μM) |
| 10 | 5 | 5 | 1 | 1 |

After the second PCR, we analyzed the DNA via 1% agarose gel electrophoresis and excised products of the expected size from the gel. When we were unable to obtain a discrete product band, we re-ran the Perl-mTAS program using a modified gap length as an input, and this provided us an improved mTAS oligonucleotide set. These redesigned sets included 9-1, 9-2, 9long, 10-2, 10long, 11-1, 11-2, 11long, 12, 13, and 19 set (Table 10).

We purified the amplified products using the AccuPrep™ gel purification kit (Bioneer, Korea), cloned them into a vector (pTWIN1; New England Biolab) using a restriction enzyme (Fermentas), and used them to transform competent E. coli cells. The restriction enzyme sites are summarized in Tables 1-9. After overnight growth, randomly selected colonies were screened by colony PCR to confirm correct insertion of the amplified products. Appropriate colonies were transferred to Luria-Bertani broth (BD Science) containing carbenicillin (Sigma-Aldrich) and were then sent for sequencing by using a primer (5′-GAAGAAGGTAAACTGACAAATCC-3′) (SEQ ID No: 215) after plasmid extraction using the AccuPrep™ plasmid extraction kit (Bioneer, Korea). Resulting sequencing data were analyzed using Lasergene (DNAstar, Madison, Wis.).

mTAS Target Sequencing Using Genomic DNA Purified from Lung Cancer Tissues

We prepared genomic DNA from both lung cancer tumor tissue and normal tissue using the AccuPrep™ Genomic DNA Extraction Kit (Bioneer, Korea). The mTAS condition was identical to that described above. After the second PCR, we ran agarose (Bioneer) gel electrophoresis and excised the desired products. We sent the gel-purified DNA samples for Sanger sequencing. We analyzed the sequencing data using Lasergene (DNAstar, Madison, Wis.).

### Results and Discussion

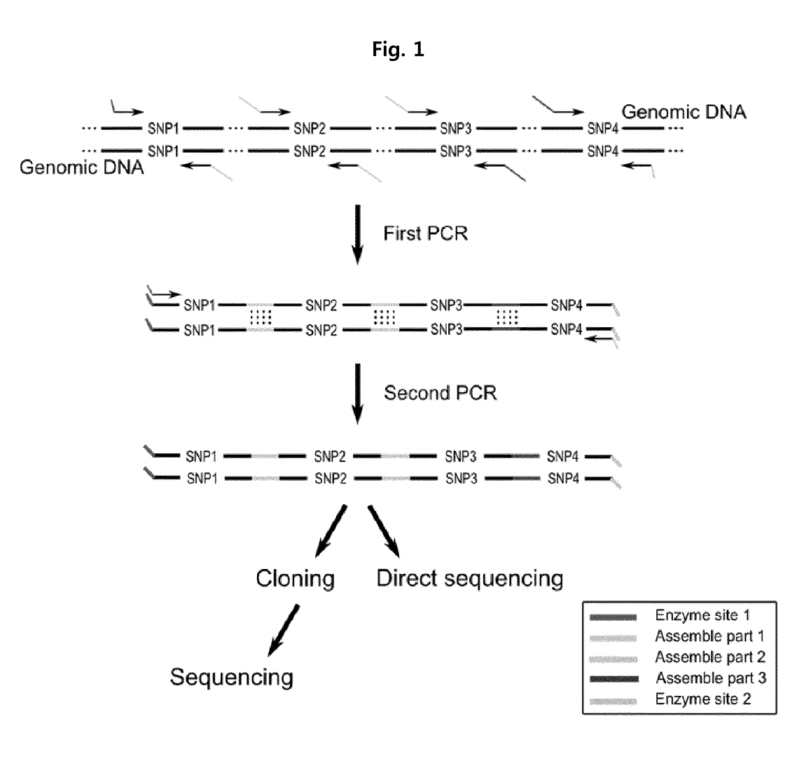

The mTAS method takes advantage of polymerase cycling assembly (PCA) (17), a method to construct large stretches of DNA. The PCA method typically uses multiple overlapping oligonucleotides that are designed to assemble via a polymerase chain reaction. For mTAS target sequencing, we designed multiple PCA probes, each having target-specific sequences at the 3′-end and assembly spacer sequences at the 5′-end (FIG. 1). These probes first generate multiple short amplicons, and in the second round of the assembly process, the overlapping spacer sequences are used to assemble short amplicons into a large stretch of the desired DNA sequence.

To test the utility of mTAS for targeted sequencing, we assembled various sets of disease- and specific phenotype-related human SNPs (18) from those listed on the website of a commercial genetic testing service (https://www.23andme.com/). The selected SNP sequences are shown in Tables 1-9. To facilitate the design of oligonucleotides for the mTAS experiments, we developed a Perl program, Perl-mTAS. Briefly, Perl-mTAS generates overlapping oligonucleotides optimized for certain input parameters, which include a SNP ID, the target locus length, and the oligo assembly temperature. We used the nearest neighbor method to calculate the assembly temperatures for regions overlapping adjacent oligonucleotides (19). The oligonucleotide sequences generated from the Perl-mTAS are listed in Tables 1-9.

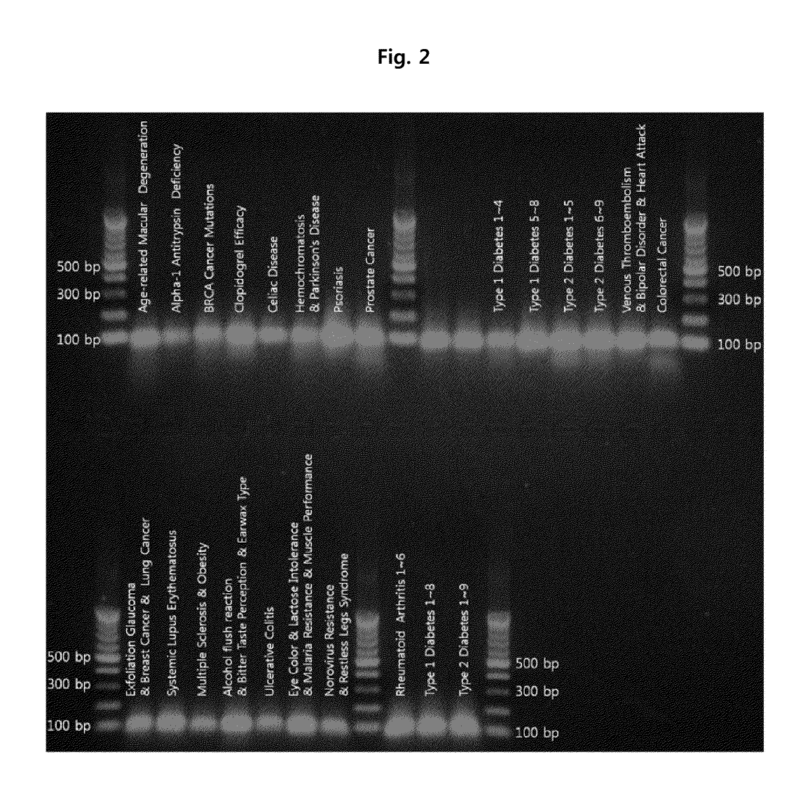

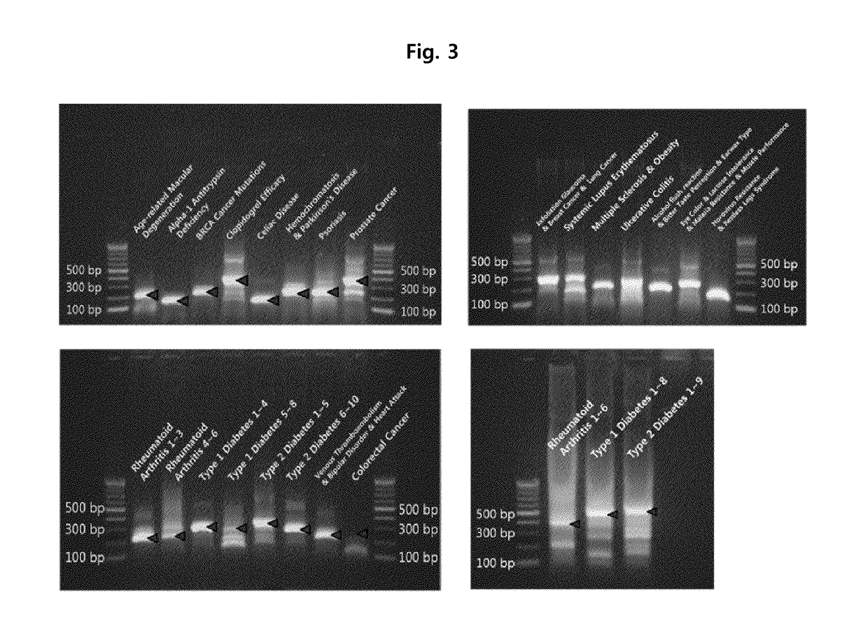

The assembly process for mTAS proceeds in two steps. We used genomic DNA purified from human blood. The first assembly step generated a mixture of amplicons of about 100 bp (FIG. 2). We then mixed an aliquot of the first amplification products, without further purification, into an excess pair of flanking primer oligonucleotides to begin a second assembly process. Using an optimized protocol for the assembly process (See, Methods), we were able to assemble 25 amplicons out of mTAS experimental sets (FIG. 3). We found that the concentration of oligonucleotides used for the first and the second assembly steps are important to obtain the desired amplicons as major products (See, Methods and Tables 10-11). We also found that in the majority of experiments, the assembly of two to five SNPs proceeded with high efficiency, as shown in FIG. 1. We grouped the two to five SNPs based on the phenotypes. For example, we used all of the SNP loci listed for AMD (Age-related Macular Degeneration) on https://www.23andme.com/ as one set. For phenotypes that contain only one SNP, we put a few of these SNPs together to carry out one mTAS experiment. Notably, we achieved mTAS target sequencing of six to nine loci, as illustrated for Rheumatoid Arthritis (six SNPs), Type 1 Diabetes (eight SNPs), and Type 2 Diabetes (nine SNPs) (FIG. 2). However, we found that the amplifications were less efficient in these experiments. Thus, when the number of SNPs is more than five, we may need to divide these mTAS experiments into two sets.

To characterize the mTAS method in detail, we cloned the amplicons and used Sanger sequencing to confirm sequences of captured target loci. We found that the majority of the target sequences were perfectly assembled with only a few exceptions, which led to the loss of some target loci from assembly amplicons (Tables 12-16).

TABLE 12
Sanger sequencing result from mTAS.

| | | | SNP | SNP | SNP | SNP | SNP |
|---|---|---|---|---|---|---|---|
| set | | | No. 1 | No. 2 | No. 3 | No. 4 | No. 5 |
| 1 set | | SNP site | rs1061147 | rs547154 | rs3750847 | | |
| | | Reference | A | G | C | | |
| | 1^{st} | Seq. | C, C, C, C | G, G, G, G | C, C, C, C | | |
| | experiment | result | | | | | |
| | 2^{nd} | Seq. | C, C, C | G, G, G | C, C, C, | | |
| | experiment | result | | | | | |
| | 3^{rd} | Seq. | C, C, C | G, G, G | C, C, C | | |
| | experiment | result | | | | | |
| 2 set | | SNP site | rs17580 | Rs28929474 | | | |
| | | Reference | T | C | | | |
| | 1^{st} | Seq. | T, T, T | C, C, C | | | |
| | experiment | result | | | | | |
| | 2^{nd} | Seq. | T, T, T | C, C, C | | | |
| | experiment | result | | | | | |
| | 3^{rd} | Seq. | T, T, T | C, C, C | | | |
| | experiment | result | | | | | |
| 3 set | | SNP site | 185delAG | 5382insC | 6174delT | | |
| | | Reference | CT | T | A | | |
| | 1^{st} | Seq. | CT, CT, CT, | T, T, T, T | A, A, A, A | | |
| | experiment | result | CT | | | | |
| | 2^{nd} | Seq. | CT, CT, CT, | T, T, T, T | A, A, A, A | | |
| | experiment | result | CT | | | | |
| | 3^{rd} | Seq. | CT, CT, CT, | T, T, T, T | A, A, A, A | | |
| | experiment | result | CT | | | | |
| 4 set | | SNP site | rs4244285 | rs4986893 | rs28399504 | rs41291556 | rs12248560 |
| | | Reference | G | G | A | T | C |
| | 1^{st} | Seq. | G, G | G, G | A, A | T, T | C, C |
| | experiment | result | | | | | |
| | 2^{nd} | Seq. | G, G, G | G, G/A | A, A, A | T, T, T | C, C, C |
| | experiment | result | | | | | |
| | 3^{rd} | Seq. | G, G, G | A, A/G | A, A, A | T, T, T | C, C, C |
| | experiment | result | | | | | |

For each set, we carried out three repeat experiments (shown as 1^{st}, 2^{nd}, and 3^{rd} set) For each set, we sent multiple colonies for sequencing (four colonies in 1^{st} set; and three colonies in 2^{nd} and 3^{rd} set); each sequencing data are listed with comma.

TABLE 13
Sanger sequencing result from mTAS.

| | | | | | | |
|---|---|---|---|---|---|---|
| | SNP | SNP | SNP | SNP | SNP | SNP |
| set | No. 1 | No. 2 | No. 3 | No. 4 | No. 5 | No. 6 |

| | | | | | | | |
|---|---|---|---|---|---|---|---|
| 6 set | | SNP | rs1800562 | rs1799945 | rs34637584 | | |
| | | site | | | | | |
| | | Reference | G | C | G | | |
| | 1^{st} | Seq. | G, G, G, G | C, C, C, C | G, G, G, G | | |
| | experiment | result | | | | | |
| | 2^{nd} | Seq. | G, G, G, G | C, C, C, C | G, G, G, G | | |
| | experiment | result | | | | | |
| | 3^{rd} | Seq. | G, G, G, G | C, C, C, C | G, G, G, G | | |
| | experiment | result | | | | | |
| 7 set | | SNP | rs10484554 | rs3212227 | rs11209026 | | |
| | | site | | | | | |
| | | Reference | C | T | G | | |
| | 1^{st} | Seq. | C, C, C, C | G, G, G, G | G, G, G, G | | |
| | experiment | result | | | | | |
| | 2^{nd} | Seq. | C, C, C | G, G, G | G, G, G | | |
| | experiment | result | | | | | |
| | 3^{rd} | Seq. | C, C, C | G, G, G | G, G, G | | |
| | experiment | result | | | | | |
| 8 set | | SNP | rs1447295 | rs6983267 | rs10505483 | rs1859962 | rs4430796 |
| | | site | | | | | |
| | | Reference | A | G | C | G | C |
| | 1^{st} | Seq. | C/A | T, T | T, T | G, G | G, G |
| | experiment | result | | | | | |
| | 2^{nd} | Seq. | C, C/A | T, T, T | T, T, T | G, G, G | A, A, A |
| | experiment | result | | | | | |
| | 3^{rd} | Seq. | A/C | T, T | T, T | G, G | G/A |
| | experiment | result | | | | | |
| 9-1 | | SNP | rs6457617 | rs11203366 | rs2476601 | | |
| set | | site | | | | | |
| | | Reference | C | G | A | | |
| | 1^{st} | Seq. | T, T/C | A, A/G | G, G, G | | |
| | experiment | result | | | | | |
| | 2^{nd} | Seq. | C, C/T | A, A, A | G, G, G | | |
| | experiment | result | | | | | |
| | 3^{rd} | Seq. | T, T/C | A, A/G | G, G, G | | |
| | experiment | result | | | | | |
| 9-2 | | SNP | rs3890745 | rs2327832 | rs3761847 | | |
| set | | site | | | | | |
| | | Reference | T | A | G | | |
| | 1^{st} | Seq. | C, C/T | A, A, A | A, A/G | | |
| | experiment | result | | | | | |
| | 2^{nd} | Seq. | C, C/T | A, A, A | G, G/A | | |
| | experiment | result | | | | | |
| | 3^{rd} | Seq. | C, C, C | A, A, A | A, A/G | | |
| | experiment | result | | | | | |

For each set, we carried out three repeat experiments (shown as 1^{st}, 2^{nd}, and 3^{rd} set) For each set, we sent multiple colonies for sequencing (four colonies in 1^{st} set; and three colonies in 2^{nd} and 3^{rd} set); each sequencing data are listed with comma.

TABLE 14
Sanger sequencing result from mTAS.

| | | | SNP | SNP | SNP | SNP | SNP |
|---|---|---|---|---|---|---|---|
| set | | | No. 1 | No. 2 | No. 3 | No. 4 | No. 5 |
| 9 long | | SNP | Rs6457617 | Rs11203366 | Rs2476601 | Rs3890745 | Rs2327832 |
| set | | site | | | | | |
| | | Reference | C | G | A | T | A |
| | 1^{st} | Seq. | C, C | A, A | G, G | C, C | A/C |
| | experiment | result | | | | | |
| | 2^{nd} | Seq. | C/T | A/G | G, G | C, C | A, A |
| | experiment | result | | | | | |
| | 3^{rd} | Seq. | x, x | x, x | x, x | x, x | |
| | experiment | result | | | | | |
| 10-1 | | SNP | rs3129934 | rs3087243 | rs1990760 | rs3741208 | |
| set | | site | | | | | |
| | | Reference | T | G | C | A | |
| | 1^{st} | Seq. | x, x, x | G, G, G | C, C, C | G, | |
| | experiment | result | | | | G/A | |
| | 2^{nd} | Seq. | x, x, x | G, G, G | C, C, C | G, G, G | |
| | experiment | result | | | | | |
| | 3^{rd} | Seq. | x, x | G, G | C, C | A, A | |
| | experiment | result | | | | | |
| 10-2 | | SNP | rs1893217 | rs2476601 | rs3184504 | rs725613 | |
| set | | site | | | | | |
| | | Reference | A | A | T | T | |
| | 1^{st} | Seq. | x, x, x | x, x, x | C, C, C | G, G, G | |
| | experiment | result | | | | | |
| | 2^{nd} | Seq. | x, x, x | x, x, x | T, T/C | G, G, G | |
| | experiment | result | | | | | |
| | 3^{rd} | Seq. | x, x | x, x | C/T | G, G | |
| | experiment | result | | | | | |
| 10 long | | SNP | rs3129934 | rs3087243 | rs1990760 | rs3741208 | rs1893217 |
| set | | site | | | | | |
| | | Reference | T | G | C | A | A |
| | 1^{st} | Seq. | C, C, C | G, G, G | C, C, C | A, A, A | G/A, x |
| | experiment | result | | | | | |
| | 2^{nd} | Seq. | C, C, C | G, G, G | C, C, C | G, G, G | G, |
| | experiment | result | | | | | G/A |
| | 3^{rd} | Seq. | C, C, C | G, G, G | C, C, C | G, | A, A, A |
| | experiment | result | | | | G/A | |
| 11-1 | | SNP | rs7903146 | rs1801282 | rs5219 | rs4402960 | rs1111875 |
| set | | site | | | | | |
| | | Reference | C | C | T | G | A |
| | 1^{st} | Seq. | C, C, C | C, C, C | T, T, C | G, G, G | T, T, T |
| | experiment | result | | | | | |
| | 2^{nd} | Seq. | C, C | C, C | C, C | G, G | C, T |
| | experiment | result | | | | | |
| | 3^{rd} | Seq. | C, C/x | C, C, C | C, T, T | G, G, G | C/x, x |
| | experiment | result | | | | | |

| | | | SNP | SNP | SNP | SNP |
|---|---|---|---|---|---|---|
| set | | | No. 6 | No. 7 | No. 8 | No. 9 |
| 9 long | | SNP | Rs3761847 | | | |
| set | | site | | | | |
| | | Reference | G | | | |
| | 1^{st} | Seq. | A/G | | | |
| | experiment | result | | | | |
| | 2^{nd} | Seq. | A/G | | | |
| | experiment | result | | | | |
| | 3^{rd} | Seq. | | | | |
| | experiment | result | | | | |
| 10-1 | | SNP | | | | |
| set | | site | | | | |
| | | Reference | | | | |
| | 1^{st} | Seq. | | | | |
| | experiment | result | | | | |
| | 2^{nd} | Seq. | | | | |
| | experiment | result | | | | |
| | 3^{rd} | Seq. | | | | |
| | experiment | result | | | | |
| 10-2 | | SNP | | | | |
| set | | site | | | | |
| | | Reference | | | | |
| | 1^{st} | Seq. | | | | |
| | experiment | result | | | | |
| | 2^{nd} | Seq. | | | | |
| | experiment | result | | | | |
| | 3^{rd} | Seq. | | | | |
| | experiment | result | | | | |
| 10 long | | SNP | rs2476601 | rs3184504 | rs725613 | |
| set | | site | | | | |
| | | Reference | A | T | T | |
| | 1^{st} | Seq. | G/x, x | T/x, x | G/x, x | |
| | experiment | result | | | | |
| | 2^{nd} | Seq. | G, G, G | C, C, C | G, | |
| | experiment | result | | | G/T | |
| | 3^{rd} | Seq. | G, G, G | C, C, C | G, G, G | |
| | experiment | result | | | | |
| 11-1 | | SNP | | | | |
| set | | site | | | | |
| | | Reference | | | | |
| | 1^{st} | Seq. | | | | |
| | experiment | result | | | | |
| | 2^{nd} | Seq. | | | | |
| | experiment | result | | | | |
| | 3^{rd} | Seq. | | | | |
| | experiment | result | | | | |

For each set, we carried out three repeat experiments (shown as 1^{st}, 2^{nd}, and 3^{rd} set) For each set, we sent multiple colonies for sequencing (four colonies in 1^{st} set; and three colonies in 2^{nd} and 3^{rd} set); each sequencing data are listed with comma.

TABLE 15
Sanger sequencing result from mTAS.

| | | | SNP | SNP | SNP | SNP | SNP | SNP | | | |
|---|---|---|---|---|---|---|---|---|---|---|---|
| set | | | No. 1 | No. 2 | No. 3 | No. 4 | No. 3 | No. 4 | | | |
| 11-2 | | SNP | rs4712523 | rs13266634 | rs10012946 | rs2383208 | | | | | |
| set | | site | | | | | | | | | |
| | | Reference | A | C | T | A | | | | | |
| | 1^{st} | Seq. | G, | C, | C, | A, | | | | | |
| | experiment | result | G/A | T/x | C/T | A/x | | | | | |
| | 2^{nd} | Seq. | G, G, G | C, C, C | C, C, C | A, A, A | | | | | |
| | experiment | result | | | | | | | | | |
| | 3^{rd} | Seq. | G, G, G | T, T/C | C, C, C | A, A, A | | | | | |
| | experiment | result | | | | | | | | | |
| 11 | | SNP | rs7903146 | rs1801282 | rs5219 | rs4402960 | rs1111875 | rs4712523 | rs13266634 | rs10012946 | rs2383208 |
| long | | site | | | | | | | | | |
| set | | Reference | C | C | T | G | C | A | C | T | A |
| | 1^{st} | Seq. | C, C, C | C, C, C | C, | G, | T, T, T | G, G, G | C, | C, C, C | A, A, A |
| | experiment | result | | | C/T | G/T | | | C/T | | |
| | 2^{nd} | Seq. | C, C, C | C, C, C | T, | G, | C, | G, | T, | C, C, C | A, |
| | experiment | result | | | T/C | G/T | C/T | G/A | T/C | | A/C |
| | 3^{rd} | Seq. | C, C, C | C, C, C | T, | G, G, G | T, | G, G, G | C, | C, C, C | A, A, A |
| | experiment | result | | | T/C | | T/C | | C/T | | |
| 12 | | SNP | rs6025 | rs4948418 | | | | | | | |
| set | | site | | | | | | | | | |
| | | Reference | T | C | | | | | | | |
| | 1^{st} | Seq. | C, C, C | C, C, C | | | | | | | |
| | experiment | result | | | | | | | | | |
| | 2^{nd} | Seq. | C, C | C, C | | | | | | | |
| | experiment | result | | | | | | | | | |
| | 3^{rd} | Seq. | C, C, C | C, C, C | | | | | | | |
| | experiment | result | | | | | | | | | |
| 13 | | SNP | rs6983267 | rs4939827 | | | | | | | |
| set | | site | | | | | | | | | |
| | | Reference | G | T | | | | | | | |
| | 1^{st} | Seq. | T, T | C, C | | | | | | | |
| | experiment | result | | | | | | | | | |
| | 2^{nd} | Seq. | x, x | x, x | | | | | | | |
| | experiment | result | | | | | | | | | |
| | 3^{rd} | Seq. | x, x | x, x | | | | | | | |
| | experiment | result | | | | | | | | | |
| 14 | | SNP | rs2165241 | rs1219648 | rs8034191 | | | | | | |
| set | | site | | | | | | | | | |
| | | Reference | T | A | T | | | | | | |
| | 1^{st} | Seq. | C, C | G, G | T, T | | | | | | |
| | experiment | result | | | | | | | | | |
| | 2^{nd} | Seq. | C, C, C | G, | T, T, T | | | | | | |
| | experiment | result | | G/A | | | | | | | |
| | 3^{rd} | Seq. | C, C, C | A, | T, T, T | | | | | | |
| | experiment | result | | A/G | T | | | | | | |

For each set, we carried out three repeat experiments (shown as 1^{st}, 2^{nd}, and 3^{rd} set) For each set, we sent multiple colonies for sequencing (four colonies in 1^{st} set; and three colonies in 2^{nd} and 3^{rd} set); each sequencing data are listed with comma.

TABLE 16
Sanger sequencing result from mTAS.

| set | | | SNP No. 1 | SNP No. 2 | SNP No. 3 | SNP No. 4 |
|---|---|---|---|---|---|---|
| 15 | | SNP site | rs9888739 | rs7574865 | rs10488631 | |
| set | | Reference | C | T | T | |
| | 1^{st} | Seq. | C, C, C, C | G, G, G/T | T, T, T, T | |
| | experiment | result | | | | |
| | 2^{nd} | Seq. | C, C, C | T, T/G | T, T, T | |
| | experiment | result | | | | |
| | 3^{rd} | Seq. | C, C, C | G, G, G | T, T, T | |
| | experiment | result | | | | |
| 16 | | SNP site | rs6897932 | rs3135388 | | |
| set | | Reference | C | A | | |
| | 1^{st} | Seq. | T, T, T/C | G, G, G, G | | |
| | experiment | result | | | | |
| | 2^{nd} | Seq. | T, T/C | G, G, G | | |
| | experiment | result | | | | |
| | 3^{rd} | Seq. | T, T, T | G, G, G | | |
| | experiment | result | | | | |
| 17 | | SNP site | rs2395185 | rs9858542 | rs10883365 | rs11209026 |
| set | | Reference | G | G | G | G |
| | 1^{st} | Seq. | G, G, G, G | G, G, G, G | G, G, G, G | G, G, G, G |
| | experiment | result | | | | |
| | 2^{nd} | Seq. | G, G, G | G, G, G | G, G, G | G, G, G |
| | experiment | result | | | | |
| | 3^{rd} | Seq. | G, G, G | G, G, G | G, G, G | G, G, G |
| | experiment | result | | | | |
| 18 | | SNP site | rs671 | rs713598 | rs17822931 | |
| set | | Reference | G | C | C | |
| | 1^{st} | Seq. | G, G, G, G | C, C, C/G | T, T, T, T | |
| | experiment | result | | | | |
| | 2^{nd} | Seq. | G, G, G | C, C, C | T, T, T | |
| | experiment | result | | | | |
| | 3^{rd} | Seq. | G, G, G | C, C/G | T, T, T | |
| | experiment | result | | | | |
| 19 | | SNP site | rs12913832 | rs4988235 | rs2814778 | rs1815739 |
| set | | Reference | A | G | T | C |
| | 1^{st} | Seq. | A | G | T | C |
| | experiment | result | | | | |
| | 2^{nd} | Seq. | A, A, A | G, G, G | T, T, T | C, C, C |
| | experiment | result | | | | |
| | 3^{rd} | Seq. | A, A, A | G, G, G | T, T, T | C, C, C |
| | experiment | result | | | | |
| 20 | | SNP site | rs601338 | rs3923809 | | |
| set | | Reference | G | A | | |
| | 1^{st} | Seq. | G, G, G | G, G, G | | |
| | experiment | result | | | | |
| | 2^{nd} | Seq. | G, G, G | G, G, G | | |
| | experiment | result | | | | |
| | 3^{rd} | Seq. | G, G, G | G, G, G | | |
| | experiment | result | | | | |

For each set, we carried out three repeat experiments (shown as 1^{st}, 2^{nd}, and 3^{rd} set) For each set, we sent multiple colonies for sequencing (four colonies in 1^{st} set; and three colonies in 2^{nd} and 3^{rd} set); each sequencing data are listed with comma.

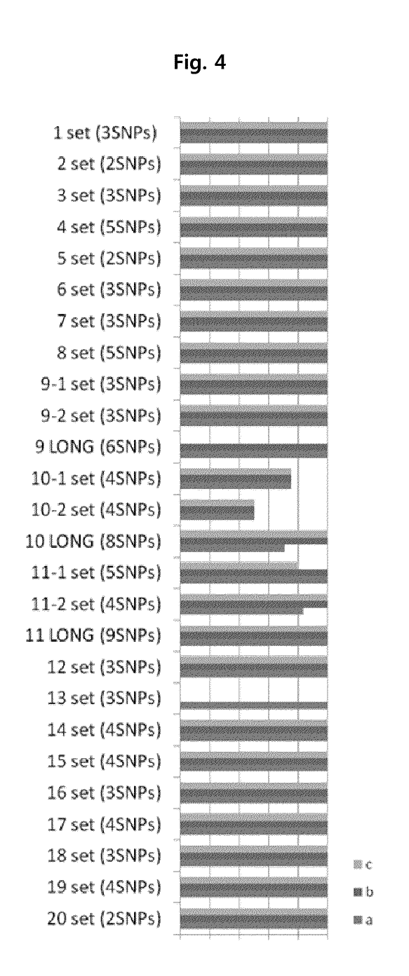

We repeated the assembly of 20 amplicons three more times and found that the assembly efficiency was comparable to the efficiency level of the first experiments (FIG. 4 and Tables 14-16). In these experiments, we used cloning procedures to evaluate mTAS precisely; however, the PCR products from mTAS can be sequenced directly after agarose gel purification, as discussed below.

Mutations in the epidermal growth factor receptor (EGFR) are a leading cause of a non-small cell lung cancer (NSCLC) (16). More than 90% of EGFR mutations are present in exon 19 (five amino acid deletion mutation) and in exon 21 (Leu858Arg mutation from a single nucleotide change). More importantly, tyrosine kinase inhibitor drugs (gefitinib and erlotinib) targeting these EGFR mutations develop a drug resistance cancer mainly from the emergence of an exon 20 mutation (Thr790Met). At present, because the identification of these EGFR mutations is very important for screening patients for the personalized therapy, lung cancer patient's tumor tissue samples are often examined by PCR assessments of these loci followed by multiple Sanger sequencing runs.

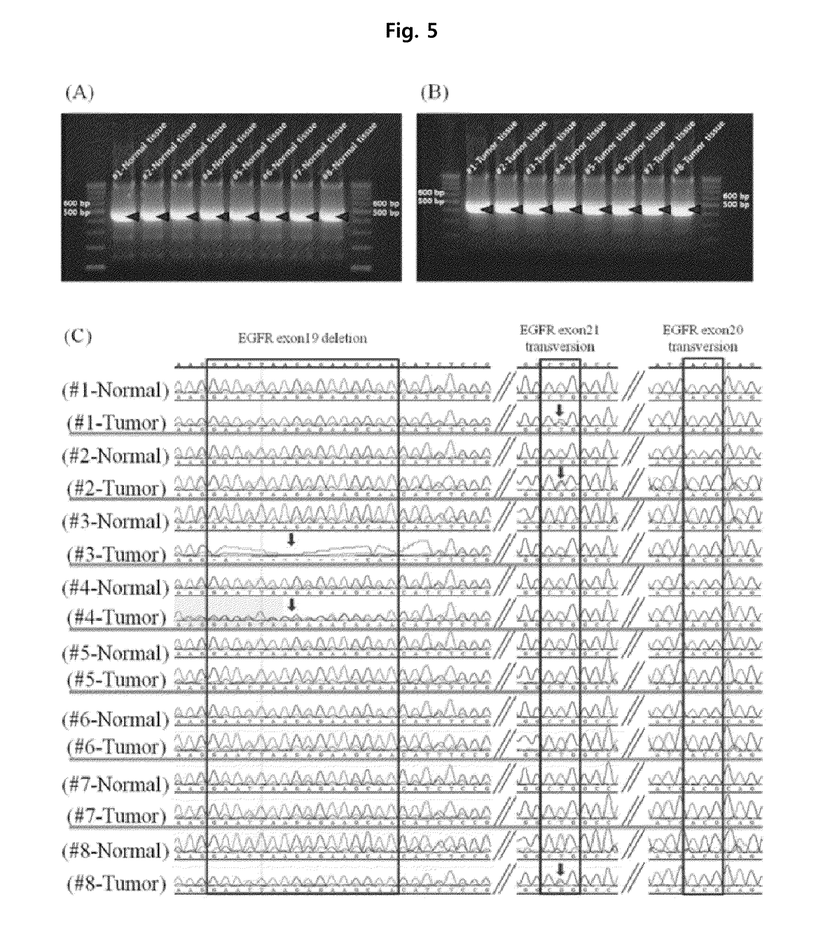

By applying mTAS sequencing for these clinically important EGFR target sequences, we expected to reduce the DNA sequencing cost considerably through the use of mTAS primer pairs designed for EGFR. Using genomic DNA extracted from human lung cancer tissues, we carried out mTAS target amplification of three loci, covering parts of exons 19, 20 and 21 (FIG. 5). Subsequently, we used direct Sanger sequencing of these amplicons to verify the target sequences. We successfully detected EGFR mutations related to lung cancer (arrows in FIG. 5), and our results were comparable to the sequencing results from a conventional EGFR DNA sequencing provider.

In summary, using multiple PCR primer pairs that could anneal to target genomic loci, we were able to collect the information of these loci from a single DNA sequencing run. Furthermore, mTAS target sequencing provides homogeneous enrichment over multiple target loci (about 10 loci) and results in specific and uniform evaluations of target loci. As a result, the mTAS target sequencing process provides a unique solution for cost-effective analyses of clinical samples that are typically examined by Sanger sequencing runs. Currently, most clinical genetic tests are carried out using Sanger sequencing. Thus, by amplifying these multiple clinical genetic test target loci in one sequence read, we can reduce the cost of Sanger sequencing many folds. Although we used Sanger sequencing here to evaluate mTAS, this method can be used in conjunction with high-throughput sequencing technology to increase the throughput even further. For example, the Roche-454 sequencing platform, which has a read length of about 500 bp, can be used with mTAS to detect single-nucleotide polymorphisms (SNP) spread out over the genome while retaining most of the sequence data.

Having described a preferred embodiment of the present invention, it is to be understood that variants and modifications thereof falling within the spirit of the invention may become apparent to those skilled in this art, and the scope of this invention is to be determined by appended claims and their equivalents.

### REFERENCES

1. Yang, S. and Rothman, R. E. (2004) PCR-based diagnostics for infectious diseases: uses, limitations, and future applications in acute-care settings. Lancet Infect Dis, 4, 337-348.
2. Mamanova, L., Coffey, A. J., Scott, C. E., Kozarewa, I., Turner, E. H., Kumar, A., Howard, E., Shendure, J. and Turner, D. J. (2010) Target-enrichment strategies for next-generation sequencing. Nat Methods, 7, 111-118.
3. Saiki, R. K., Gelfand, D. H., Stoffel, S., Scharf, S. J., Higuchi, R., Horn, G. T., Mullis, K. B. and Erlich, H. A. (1988) Primer-directed enzymatic amplification of DNA with a thermostable DNA polymerase. Science, 239, 487-491.
4. Edwards, M. C. and Gibbs, R. A. (1994) Multiplex PCR: advantages, development, and applications. PCR Methods Appl, 3, S65-75.
5. Chun, J. Y., Kim, K. J., Hwang, I. T., Kim, Y. J., Lee, D. H., Lee, I. K. and Kim, J. K. (2007) Dual priming oligonucleotide system for the multiplex detection of respiratory viruses and SNP genotyping of CYP2C19 gene. Nucleic Acids Res, 35, e40.
6. Lovett, M., Kere, J. and Hinton, L. M. (1991) Direct selection: a method for the isolation of cDNAs encoded by large genomic regions. Proc Natl Acad Sci USA, 88, 9628-9632.
7. Parimoo, S., Patanjali, S. R., Shukla, H., Chaplin, D. D. and Weissman, S. M. (1991) cDNA selection: efficient PCR approach for the selection of cDNAs encoded in large chromosomal DNA fragments. Proc Natl Acad Sci USA, 88, 9623-9627.
8. Albert, T. J., Molla, M. N., Muzny, D. M., Nazareth, L., Wheeler, D., Song, X., Richmond, T. A., Middle, C. M., Rodesch, M. J., Packard, C. J. et al. (2007) Direct selection of human genomic loci by microarray hybridization. Nat Methods, 4, 903-905.
9. Gnirke, A., Melnikov, A., Maguire, J., Rogov, P., LeProust, E. M., Brockman, W., Fennell, T., Giannoukos, G., Fisher, S., Russ, C. et al. (2009) Solution hybrid selection with ultra-long oligonucleotides for massively parallel targeted sequencing. Nat Biotechnol, 27, 182-189.
10. Lizardi, P. M., Huang, X., Zhu, Z., Bray-Ward, P., Thomas, D. C. and Ward, D. C. (1998) Mutation detection and single-molecule counting using isothermal rolling-circle amplification. Nat Genet, 19, 225-232.
11. Antson, D. O., Isaksson, A., Landegren, U. and Nilsson, M. (2000) PCR-generated padlock probes detect single nucleotide variation in genomic DNA. Nucleic Acids Res, 28, E58.
12. Hardenbol, P., Baner, J., Jain, M., Nilsson, M., Namsaraev, E. A., Karlin-Neumann, G. A., Fakhrai-Rad, H., Ronaghi, M., Willis, T. D., Landegren, U. et al. (2003) Multiplexed genotyping with sequence-tagged molecular inversion probes. Nat Biotechnol, 21, 673-678.
13. Hardenbol, P., Yu, F., Belmont, J., Mackenzie, J., Bruckner, C., Brundage, T., Boudreau, A., Chow, S., Eberle, J., Erbilgin, A. et al. (2005) Highly multiplexed molecular inversion probe genotyping: over 10,000 targeted SNPs genotyped in a single tube assay. Genome Res, 15, 269-275.
14. Tewhey, R., Warner, J. B., Nakano, M., Libby, B., Medkova, M., David, P. H., Kotsopoulos, S. K., Samuels, M. L., Hutchison, J. B., Larson, J. W. et al. (2009) Microdroplet-based PCR enrichment for large-scale targeted sequencing. Nat Biotechnol, 27, 1025-1031.
15. Shendure, J. and Ji, H. (2008) Next-generation DNA sequencing. Nat Biotechnol, 26, 1135-1145.
16. Sharma, S. V., Bell, D. W., Settleman, J. and Haber, D. A. (2007) Epidermal growth factor receptor mutations in lung cancer. Nat Rev Cancer, 7, 169-181.
17. Stemmer, W. P., Crameri, A., Ha, K. D., Brennan, T. M. and Heyneker, H. L. (1995) Single-step assembly of a gene and entire plasmid from large numbers of oligodeoxyribonucleotides. Gene, 164, 49-53.
18. Carlson, B. (2008) SNPs—A shortcut to personalized medicine. Genet Eng Biotechn N, 28, 12-12.
19. SantaLucia, J., Jr. (1998) A unified view of polymer, dumbbell, and oligonucleotide DNA nearest-neighbor thermodynamics. Proc Natl Acad Sci USA, 95, 1460-1465.

1. A method for assembling multiple target loci into a single shortened nucleic acid sequence, comprising:
(a) obtaining a target nucleic acid molecule including multiple target loci including at least two target loci on one molecule thereof;
(b) obtaining primary amplification products by primary amplification of the target nucleic molecule using a primary amplification primer set including at least two primer pairs for being hybridized with upstream and downstream regions of the at least two target loci and amplifying flanking regions of the at least two target loci, wherein the at least two primer pairs each having a forward primer and a reverse primer and the at least two primer pairs include a first primer pair for amplifying a first target locus which is located relatively in the 5′ direction and a second primer pair for amplifying a second target locus which is located in the 3′ direction of the first target locus; wherein a reverse primer of the first primer pair includes (i) a target hybridization nucleotide sequence that is complementary to a downstream region of the first target locus and (ii) an overlapping sequence that is non-complementary to the target nucleic acid molecule but complementary to a forward primer of the second primer pair; and wherein the forward primer of the second primer pair includes (i) a target hybridization nucleotide sequence that is complementary to an upstream region of the second target locus and (ii) an overlapping sequence that is non-complementary to the target nucleic acid molecule but complementary to the reverse primer of the first primer pair; and
(c) obtaining secondary amplification products by secondary amplification using a secondary amplification primer set and the primary amplification products, the secondary amplification primer set including a primer that is complementary to a 5′ end region formed when the primary amplification products are arranged in the 5′ to 3′ direction and a primer that is complementary to a 3′ end region of the sequence, wherein the secondary amplification products constitute a nucleic acid sequence in which the at least two target loci are located adjacent to each other, the nucleic acid being extended to have a greater length than the target nucleic acid molecule used in step (a).

2. A method for simultaneously detecting multiple target loci, the method comprising:
analyzing the presence or absence of the at least two target loci in the secondary amplification products obtained by the method of claim 1.

3. The method of claim 1, wherein the target nucleic acid molecule includes at least three target loci and the primary amplification primer set used in the step (b) includes at least three primer pairs, the at least three primer pairs including a first primer pair for amplifying a first target locus which is located relatively farthest in the 5′ direction, a second primer pair for amplifying a second target locus which is located in the 3′ direction of the first target locus, and a third primer pair for amplifying a third target locus which is located in the 3′ direction of the second target locus; wherein a reverse primer of the first primer pair includes (i) a target hybridization nucleotide sequence that is complementary to a downstream region of the first target locus and (ii) an overlapping sequence that is non-complementary to the target nucleic acid molecule but complementary to a forward primer of the second primer pair; wherein the forward primer of the second primer pair includes (i) a target hybridization nucleotide sequence that is complementary to an upstream region of the second target locus and (ii) an overlapping sequence that is non-complementary to the target nucleic acid molecule but complementary to the reverse primer of the first primer pair, and a reverse of the second primer pair includes (i) a target hybridization nucleotide sequence that is complementary to a downstream region of the second target locus and (ii) an overlapping sequence that is non-complementary to the target nucleic acid molecule but complementary to a reverse primer of the third primer pair; and wherein a forward primer of the third primer pair includes (i) a target hybridization nucleotide sequence that is complementary to an upstream region of the third target locus and (ii) an overlapping sequence that is non-complementary to the target nucleic acid molecule but complementary to the reverse primer of the second primer pair.

4. The method of claim 3, wherein the target nucleic acid molecule includes at least four target loci and the primary amplification primer set used in the step (b) includes at least four primer pairs, the at least four primer pairs including a first primer pair for amplifying a first target locus which is located relatively farthest in the 5′ direction, a second primer pair for amplifying a second target locus which is located in the 3′ direction of the first target locus, a third primer pair for amplifying a third target locus which is located in the 3′ direction of the second target locus, and a fourth primer pair for amplifying a fourth target locus which is located in the 3′ direction of the third target locus; wherein a reverse primer of the first primer pair includes (i) a target hybridization nucleotide sequence that is complementary to a downstream region of the first target locus and (ii) an overlapping sequence that is non-complementary to the target nucleic acid molecule but complementary to a forward primer of the second primer pair; wherein the forward primer of the second primer pair includes (i) a target hybridization nucleotide sequence that is complementary to an upstream region of the second target locus and (ii) an overlapping sequence that is non-complementary to the target nucleic acid molecule but complementary to the reverse primer of the first primer pair, and a reverse of the second primer pair includes (i) a target hybridization nucleotide sequence that is complementary to a downstream region of the second target locus and (ii) an overlapping sequence that is non-complementary to the target nucleic acid molecule but complementary to a reverse primer of the third primer pair; wherein a forward primer of the third primer pair includes (i) a target hybridization nucleotide sequence that is complementary to an upstream region of the third target locus and (ii) an overlapping sequence that is non-complementary to the target nucleic acid molecule but complementary to the reverse primer of the second primer pair; and wherein a forward primer of the fourth primer pair includes (i) a target hybridization nucleotide sequence that is complementary to an upstream region of the third target locus and (ii) an overlapping sequence that is non-complementary to the target nucleic acid molecule but complementary to a reverse primer of the third primer pair.

5. The method of claim 4, wherein the target nucleic acid molecule includes at least five target loci and the primary amplification primer set used in the step (b) includes at least four primer pairs, at least four primer pairs including a first primer pair for amplifying a first target locus which is located relatively farthest in the 5′ direction, a second primer pair for amplifying a second target locus which is located in the 3′ direction of the first target locus, a third primer pair for amplifying a third target locus which is located in the 3′ direction of the second target locus, a fourth primer pair for amplifying a fourth target locus which is located in the 3′ direction of the third target locus, and a fifth primer pair for amplifying a fifth target locus which is located in the 3′ direction of the fourth target locus; wherein a reverse primer of the first primer pair includes (i) a target hybridization nucleotide sequence that is complementary to a downstream region of the first target locus and (ii) an overlapping sequence that is non-complementary to the target nucleic acid molecule but complementary to a forward primer of the second primer pair; wherein the forward primer of the second primer pair includes (i) a target hybridization nucleotide sequence that is complementary to an upstream region of the second target locus and (ii) an overlapping sequence that is non-complementary to the target nucleic acid molecule but complementary to the reverse primer of the first primer pair, and a reverse of the second primer pair includes (i) a target hybridization nucleotide sequence that is complementary to a downstream region of the second target locus and (ii) an overlapping sequence that is non-complementary to the target nucleic acid molecule but complementary to a reverse primer of the third primer pair; wherein a forward primer of the third primer pair includes (i) a target hybridization nucleotide sequence that is complementary to an upstream region of the third target locus and (ii) an overlapping sequence that is non-complementary to the target nucleic acid molecule but complementary to the reverse primer of the second primer pair; wherein a forward primer of the fourth primer pair includes (i) a target hybridization nucleotide sequence that is complementary to an upstream region of the fourth target locus and (ii) an overlapping sequence that is non-complementary to the target nucleic acid molecule but complementary to a reverse primer of the third primer pair; and wherein a forward primer of the fifth primer pair includes (i) a target hybridization nucleotide sequence that is complementary to an upstream region of the fifth target locus and (ii) an overlapping sequence that is non-complementary to the target nucleic acid molecule but complementary to a reverse primer of the fourth primer pair.

6. The method of claim 1, wherein the target nucleic acid molecule is DNA or RNA.

7. The method of claim 1, wherein the target loci are loci of nucleotide variations.

8. The method of claim 7, wherein the nucleotide variations includes single nucleotide mutation (point mutation), insertion mutation, and deletion mutation.

9. The method of claim 8, wherein the nucleotide variation is single nucleotide mutation.

10. The method of claim 1, wherein the primary amplification products in step (b) are 70˜150 bp amplicons.

11. The method of claim 2, wherein the analyzing in step (d) is performed through sequencing.

12. A kit for detecting multiple target loci, the kit comprising the primary amplification primer set and the secondary primer set of claim 1.

13. The kit of claim 12, wherein the kit is implemented by gene amplification.

14. The kit of claim 12, wherein the number of target loci is at least two.

15. The kit of claim 12, wherein the target loci are loci of nucleotide variations.

16. The kit of claim 15, wherein the nucleotide variations includes single nucleotide mutation, insertion mutation, and deletion mutation.

17. The kit of claim 16, wherein the nucleotide variation is single nucleotide mutation.

18. The kit of claim 12, wherein the detecting is performed through sequencing.


SEQUENCE LISTING (216 sequences)

SEQ ID NO: 1 - DNA, 30 bp, Artificial Sequence
  Forward Primer for rs1061147
ggtggtagatcttgcaacccggggaaatac

SEQ ID NO: 2 - DNA, 38 bp, Artificial Sequence
  Reverse primer for rs1061147
ccgttcgtcggaaaacattgccagccagtacttgtgca

SEQ ID NO: 3 - DNA, 39 bp, Artificial Sequence
  Forward primer for rs547154
caatgttttccgacgaacggttcccgcccgccagaggcc

SEQ ID NO: 4 - DNA, 36 bp, Artificial Sequence
  Reverse primer for rs547154
caccgggcacagttactggcactgtgtccaggttcc

SEQ ID NO: 5 - DNA, 47 bp, Artificial Sequence
  Forward primer for rs3750847
cagtaactgtgcccggtgtaggacatgacaagctgtcattcaagacc

SEQ ID NO: 6 - DNA, 26 bp, Artificial Sequence
  Reverse primer for rs3750847
ggtggtctcgagagccccaggcagcc

SEQ ID NO: 7 - DNA, 34 bp, Artificial Sequence
  Forward primer for Rs17580
ggtggtagatctgatgatatcgtgggtgagttca

SEQ ID NO: 8 - DNA, 39 bp, Artificial Sequence
  Reverse primer for Rs17580
tcattgtctgactggctgacgaggggaaactacagcacc

SEQ ID NO: 9 - DNA, 44 bp, Artificial Sequence
  Forward primer for Rs28929474
gtcagccagtcagacaatgactcgcccagcagcttcagtccctt

SEQ ID NO: 10 - DNA, 30 bp, Artificial Sequence
  Reverse primer for Rs28929474
ggtggtctcgagaaggctgtgctgaccatc

SEQ ID NO: 11 - DNA, 39 bp, Artificial Sequence
  Forward primer for 185delAG
ggtggtggtggtagatctttgtgctgacttaccagatgg

SEQ ID NO: 12 - DNA, 51 bp, Artificial Sequence
  Reverse primer for 185delAG
atcgattcagatgctttcacaacatgtcattaatgctatgcagaaaatctt

SEQ ID NO: 13 - DNA, 45 bp, Artificial Sequence
  Forward primer for 5382insC
gttgtgaaagcatctgaatcgatggagctttacctttctgtcctg

SEQ ID NO: 14 - DNA, 42 bp, Artificial Sequence
  Reverse primer for 5382insC
gtctcaatcgtccgaaatcttaaaaggtccaaagcgagcaag

SEQ ID NO: 15 - DNA, 45 bp, Artificial Sequence
  Forward primer for 6174delT
ttaagatttcggacgattgagaccttgtgggatttttagcacagc

SEQ ID NO: 16 - DNA, 42 bp, Artificial Sequence
  Reverse primer for 6174delT
ggtggtggtggtctcgagcatctgatacctggacagattttc

SEQ ID NO: 17 - DNA, 43 bp, Artificial Sequence
  Forward primer for rs4244285
ggtggtagatctgcaataattttcccactatcattgattattt

SEQ ID NO: 18 - DNA, 54 bp, Artificial Sequence
  Reverse primer for rs4244285
ctgggatcgattaagtaagttgaacgcaaggtttttaagtaatttgttatgggt

SEQ ID NO: 19 - DNA, 45 bp, Artificial Sequence
  Forward primer for rs4986893
gttcaacttacttaatcgatcccagatcaggattgtaagcacccc

SEQ ID NO: 20 - DNA, 41 bp, Artificial Sequence
  Reverse primer for rs4986893
ctacgaccgatcgcaatcagcaaaaaacttggccttacctg

SEQ ID NO: 21 - DNA, 47 bp, Artificial Sequence
  Forward primer for rs28399504
tgattgcgatcggtcgtagaggtaggtcttaacaagaggagaaggct

SEQ ID NO: 22 - DNA, 41 bp, Artificial Sequence
  Reverse primer for rs28399504
cctggcccttcagaggtatcgcacaaggaccacaaaaggat

SEQ ID NO: 23 - DNA, 49 bp, Artificial Sequence
  Forward primer for rs41291556
gatacctctgaagggccaggatcagggaatcgttttcagcaatggaaag

SEQ ID NO: 24 - DNA, 35 bp, Artificial Sequence
  Reverse primer for rs41291556
cgagctgatctggtggcagaaacgccggatctcct

SEQ ID NO: 25 - DNA, 50 bp, Artificial Sequence
  Forward primer for rs12248560
gccaccagatcagctcgatcagacgttcaaatttgtgtcttctgttctca

SEQ ID NO: 26 - DNA, 35 bp, Artificial Sequence
  Reverse primer for rs12248560
ggtggtctcgagggcgcattatctcttacatcaga

SEQ ID NO: 27 - DNA, 36 bp, Artificial Sequence
  Forward primer for rs2187668
ggtggtagatctaacaatcattttaccacatggtcc

SEQ ID NO: 28 - DNA, 43 bp, Artificial Sequence
  Reverse primer for rs2187668
gggcgtgggctaatgtattaaccacatatgaggcagctgagag

SEQ ID NO: 29 - DNA, 45 bp, Artificial Sequence
  Forward primer for rs6822844
gttaatacattagcccacgcccatatgtctcgctctccatagcaa

SEQ ID NO: 30 - DNA, 33 bp, Artificial Sequence
  Reverse primer for rs6822844
ggtggtctcgaggtggcaacatgaaaagagtcc

SEQ ID NO: 31 - DNA, 34 bp, Artificial Sequence
  Forward primer for rs1800562
ggtggtagatctcctggggaagagcagagatata

SEQ ID NO: 32 - DNA, 41 bp, Artificial Sequence
  Reverse primer for rs1800562
ctacggatcactcacttgtaacttagcctgggtgctccacc

SEQ ID NO: 33 - DNA, 46 bp, Artificial Sequence
  Forward primer for rs1799945
taagttacaagtgagtgatccgtaggaccagctgttcgtgttctat

SEQ ID NO: 34 - DNA, 38 bp, Artificial Sequence
  Reverse primer for rs1799945
gttttgcttcgcacaaaaagtgtccacacggcgactct

SEQ ID NO: 35 - DNA, 48 bp, Artificial Sequence
  Forward primer for rs34637586
cactttttgtgcgaagcaaaacgatccatcattgcaaagattgctgac

SEQ ID NO: 36 - DNA, 35 bp, Artificial Sequence
  Reverse primer for rs34637586
ggtggtctcgagcattctacagcagtactgagcaa

SEQ ID NO: 37 - DNA, 32 bp, Artificial Sequence
  Forward primer for rs10484554
ggtggtagatctaggtccccttcctcctatct

SEQ ID NO: 38 - DNA, 41 bp, Artificial Sequence
  Reverse primer for rs10484554
ctgggatcgattaagtaagttgaacggcaggctgagacgtc

SEQ ID NO: 39 - DNA, 50 bp, Artificial Sequence
  Forward primer for rs3212227
gttcaacttacttaatcgatcccagctgattgtttcaatgagcatttagc

SEQ ID NO: 40 - DNA, 55 bp, Artificial Sequence
  Reverse primer for rs3212227
ctacgaccgatcgcaatcagcaaaatcacaatgatatctttgctgtatttgtata

SEQ ID NO: 41 - DNA, 54 bp, Artificial Sequence
  Forward primer for rs11209026
tgattgcgatcggtcgtagaggtagttctttgattgggatatttaacagatcat

SEQ ID NO: 42 - DNA, 35 bp, Artificial Sequence
  Reverse primer for rs11209026
ggtggtctcgaggaaattctgcaaaaacctaccca

SEQ ID NO: 43 - DNA, 32 bp, Artificial Sequence
  Forward primer for rs1447295
ggtggtgaattctgccattggggaggtatgta

SEQ ID NO: 44 - DNA, 47 bp, Artificial Sequence
  Reverse primer for rs1447295
caatgtagaaagccagggtctaggttcctgttgctttttttccatag

SEQ ID NO: 45 - DNA, 45 bp, Artificial Sequence
  Forward primer for rs6983267
tagaccctggctttctacattgcaacctttgagctcagcagatga

SEQ ID NO: 46 - DNA, 48 bp, Artificial Sequence
  Reverse primer for rs6983267
tgacgggcacttagtcctcgcacataaaaattctttgtacttttctca

SEQ ID NO: 47 - DNA, 48 bp, Artificial Sequence; identical to SEQ ID NO: 46
  Forward primer for rs10505483

SEQ ID NO: 48 - DNA, 42 bp, Artificial Sequence
  Reverse primer for rs10505483
ccgagattagttctggaacgtctctgttctaaggctcatggc

SEQ ID NO: 49 - DNA, 45 bp, Artificial Sequence
  Forward primer for rs1859962
gacgttccagaactaatctcggaatacttttccaaatccctgccc

SEQ ID NO: 50 - DNA, 43 bp, Artificial Sequence
  Reverse primer for rs1859962
gcgtcagtgtgcagatcaaaatcttgggacctttaaagtgttc

SEQ ID NO: 51 - DNA, 43 bp, Artificial Sequence
  Forward primer for rs4430796
tgatctgcacactgacgccacgcggagagaggcagcacagact

SEQ ID NO: 52 - DNA, 37 bp, Artificial Sequence
  Reverse primer for rs4430796
ggtggtgcggccgctgcccaatttaagctttatgcag

SEQ ID NO: 53 - DNA, 38 bp, Artificial Sequence
  Forward primer for rs6457617
ggtggtgaattcccatatgcacagatctttgttagtca

SEQ ID NO: 54 - DNA, 40 bp, Artificial Sequence
  Reverse primer for rs6457617
tgcgtccagagagaacgtattgttgagtccatgagcagat

SEQ ID NO: 55 - DNA, 44 bp, Artificial Sequence
  Forward primer for rs11203366
tacgttctctctggacgcatgtttaggtcgtggatattgcccac

SEQ ID NO: 56 - DNA, 33 bp, Artificial Sequence
  Reverse primer for rs11203366
cccataccgctgctcgcttcttggctggagggc

SEQ ID NO: 57 - DNA, 49 bp, Artificial Sequence
  Forward primer for rs2476601
cgagcagcggtatgggccgcttcacccacaataaatgattcaggtgtcc

SEQ ID NO: 58 - DNA, 30 bp, Artificial Sequence
  Reverse primer for rs2476601
ggtggtctcgagccccctccacttcctgta

SEQ ID NO: 59 - DNA, 28 bp, Artificial Sequence
  Forward primer for rs3890745
ggtggtgaattcctgagggagggcccaa

SEQ ID NO: 60 - DNA, 40 bp, Artificial Sequence
  Reverse primer for rs3890745
cgtccatgccactgcgggggaaattgttacaaatccagac

SEQ ID NO: 61 - DNA, 59 bp, Artificial Sequence
  Forward primer for rs2327832
cgcagtggcatggacgaagataccggcacttcaataaaaaaaaattcttaaatgaaaaa

SEQ ID NO: 62 - DNA, 41 bp, Artificial Sequence
  Reverse primer for rs2327832
ggtaggcacctggcatgacatcttcagttgaggtgtccttt

SEQ ID NO: 63 - DNA, 43 bp, Artificial Sequence
  Forward primer for rs3761847
tcatgccaggtgcctaccttgtgcagtcccttctctcccctcc

SEQ ID NO: 64 - DNA, 32 bp, Artificial Sequence
  Reverse primer for rs3761847
ggtggtctcgagagagagggtggtattgaggc

SEQ ID NO: 65 - DNA, 38 bp, Artificial Sequence; identical to SEQ ID NO: 53
  Forward primer for rs6457617

SEQ ID NO: 66 - DNA, 40 bp, Artificial Sequence; identical to SEQ ID NO: 54
  Reverse primer for rs6457617

SEQ ID NO: 67 - DNA, 44 bp, Artificial Sequence; identical to SEQ ID NO: 55
  Forward primer for rs11203366

SEQ ID NO: 68 - DNA, 33 bp, Artificial Sequence; identical to SEQ ID NO: 56
  Reverse primer for rs11203366

SEQ ID NO: 69 - DNA, 49 bp, Artificial Sequence; identical to SEQ ID NO: 57
  Forward primer for rs2476601

SEQ ID NO: 70 - DNA, 43 bp, Artificial Sequence
  Reverse primer for rs2476601
ctgggatcgattaagtaagttgaacccccctccacttcctgta

SEQ ID NO: 71 - DNA, 41 bp, Artificial Sequence
  Forward primer for rs3890745
gttcaacttacttaatcgatcccagctgagggagggcccaa

SEQ ID NO: 72 - DNA, 40 bp, Artificial Sequence; identical to SEQ ID NO: 60
  Reverse primer for rs3890745

SEQ ID NO: 73 - DNA, 59 bp, Artificial Sequence; identical to SEQ ID NO: 61
  Forward primer for rs2327832

SEQ ID NO: 74 - DNA, 41 bp, Artificial Sequence; identical to SEQ ID NO: 62
  Reverse primer for rs2327832

SEQ ID NO: 75 - DNA, 43 bp, Artificial Sequence; identical to SEQ ID NO: 63
  Forward primer for rs3761847

SEQ ID NO: 76 - DNA, 32 bp, Artificial Sequence; identical to SEQ ID NO: 64
  Reverse primer for rs3761847

SEQ ID NO: 77 - DNA, 43 bp, Artificial Sequence
  Forward primer for rs3129934
ggtggtgaattctcactctcgttattctaggatacattatatt

SEQ ID NO: 78 - DNA, 37 bp, Artificial Sequence
  Reverse primer for rs3129934
cgctagctttaccgctcttccttagtgaagtggccgg

SEQ ID NO: 79 - DNA, 52 bp, Artificial Sequence
  Forward primer for rs3087243
aagagcggtaaagctagcggactgctgatttcttcaccactatttgggatat

SEQ ID NO: 80 - DNA, 45 bp, Artificial Sequence
  Reverse primer for rs3087243
tcaacctcatatggtaatcgggaggactgctatgtctgtgttaac

SEQ ID NO: 81 - DNA, 44 bp, Artificial Sequence
  Forward primer for rs1990760
cccgattaccatatgaggttgatcgtcggcacacttcttttgca

SEQ ID NO: 82 - DNA, 50 bp, Artificial Sequence
  Reverse primer for rs1990760
gaagttatgaagggtcattctgcagggaactttacattgtaagagaaaac

SEQ ID NO: 83 - DNA, 42 bp, Artificial Sequence
  Forward primer for rs3741208
gcagaatgacccttcataacttcatcggttgttgcctctccc

SEQ ID NO: 84 - DNA, 31 bp, Artificial Sequence
  Reverse primer for rs3741208
ggtggtctcgagtggacaggagactgaggag

SEQ ID NO: 85 - DNA, 33 bp, Artificial Sequence
  Forward primer for rs1893217
ggtggtgaattccacttgtcaccattcctaggg

SEQ ID NO: 86 - DNA, 42 bp, Artificial Sequence
  Reverse primer for rs1893217
ccgatgcgctggactattagatacactcttcttcctctacct

SEQ ID NO: 87 - DNA, 49 bp, Artificial Sequence
  Forward primer for rs2476601
aatagtccagcgcatcggaatgcgtccacaataaatgattcaggtgtcc

SEQ ID NO: 88 - DNA, 38 bp, Artificial Sequence
  Reverse primer for rs2476601
tttgcctaacttgcgcatttccccctccacttcctgta

SEQ ID NO: 89 - DNA, 42 bp, Artificial Sequence
  Forward primer for rs3184504
aaatgcgcaagttaggcaaacgctagcatccaggaggtccgg

SEQ ID NO: 90 - DNA, 38 bp, Artificial Sequence
  Reverse primer for rs3184504
cgtactcaaatcttaccacggttcaagccgtgtgcacc

SEQ ID NO: 91 - DNA, 47 bp, Artificial Sequence
  Forward primer for rs725613
accgtggtaagatttgagtacgttcgctgcctatcagtgtttagcac

SEQ ID NO: 92 - DNA, 29 bp, Artificial Sequence
  Reverse primer for rs725613
ggtggtctcgagatcaagacgccaggcac

SEQ ID NO: 93 - DNA, 43 bp, Artificial Sequence; identical to SEQ ID NO: 77
  Forward primer for rs3129934

SEQ ID NO: 94 - DNA, 37 bp, Artificial Sequence; identical to SEQ ID NO: 78
  Reverse primer for rs3129934

SEQ ID NO: 95 - DNA, 52 bp, Artificial Sequence; identical to SEQ ID NO: 79
  Forward primer for rs3087243

SEQ ID NO: 96 - DNA, 45 bp, Artificial Sequence; identical to SEQ ID NO: 80
  Reverse primer for rs3087243

SEQ ID NO: 97 - DNA, 44 bp, Artificial Sequence; identical to SEQ ID NO: 81
  Forward primer for rs1990760

SEQ ID NO: 98 - DNA, 50 bp, Artificial Sequence; identical to SEQ ID NO: 82
  Reverse primer for rs1990760

SEQ ID NO: 99 - DNA, 42 bp, Artificial Sequence; identical to SEQ ID NO: 83
  Forward primer for rs3741208

SEQ ID NO: 100 - DNA, 44 bp, Artificial Sequence
  Reverse primer for rs3741208
ctgggatcgattaagtaagttgaactggacaggagactgaggag

SEQ ID NO: 101 - DNA, 45 bp, Artificial Sequence
  Forward primer for rs1893217
gttcaacttacttaatcgatcccagtgtcaccattcctagggaca

SEQ ID NO: 102 - DNA, 42 bp, Artificial Sequence; identical to SEQ ID NO: 86
  Reverse primer for rs1893217

SEQ ID NO: 103 - DNA, 49 bp, Artificial Sequence; identical to SEQ ID NO: 87
  Forward primer for rs2476601

SEQ ID NO: 104 - DNA, 38 bp, Artificial Sequence; identical to SEQ ID NO: 88
  Reverse primer for rs2476601

SEQ ID NO: 105 - DNA, 42 bp, Artificial Sequence; identical to SEQ ID NO: 89
  Forward primer for rs3184504

SEQ ID NO: 106 - DNA, 38 bp, Artificial Sequence; identical to SEQ ID NO: 90
  Reverse primer for rs3184504

SEQ ID NO: 107 - DNA, 47 bp, Artificial Sequence; identical to SEQ ID NO: 91
  Forward primer for rs725613

SEQ ID NO: 108 - DNA, 29 bp, Artificial Sequence
  Reverse primer for rs725613
ggtggtctcgagatcaagacgccaggcac

SEQ ID NO: 109 - DNA, 41 bp, Artificial Sequence
  Forward primer for rs7903146
ggtggtgaattccaattagagagctaagcactttttagata

SEQ ID NO: 110 - DNA, 50 bp, Artificial Sequence
  Reverse primer for rs7903146
tcacctaggattaaccatccctgtgcctcatacggcaattaaattatata

SEQ ID NO: 111 - DNA, 48 bp, Artificial Sequence
  Forward primer for rs1801282
agggatggttaatcctaggtgacaactctgggagattctcctattgac

SEQ ID NO: 112 - DNA, 44 bp, Artificial Sequence
  Reverse primer for rs1801282
gctctggaactaaatctggacatcagtgaaggaatcgctttctg

SEQ ID NO: 113 - DNA, 41 bp, Artificial Sequence
  Forward primer for rs5219
tgtccagatttagttccagagcggagcacggtacctgggct

SEQ ID NO: 114 - DNA, 34 bp, Artificial Sequence
  Reverse primer for rs5219
acgctggccaccaatattggcagaggaccctgcc

SEQ ID NO: 115 - DNA, 50 bp, Artificial Sequence
  Forward primer for rs4402960
aatattggtggccagcgttcaaattagtaaggtaggatggacagtagatt

SEQ ID NO: 116 - DNA, 45 bp, Artificial Sequence
  Reverse primer for rs4402960
acggatgcaaagttgacgaatgtttgcaaacacaatcagtatctt

SEQ ID NO: 117 - DNA, 44 bp, Artificial Sequence
  Forward primer for rs1111875
ttcgtcaactttgcatccgttcatagagtgcaggttcagacgtc

SEQ ID NO: 118 - DNA, 35 bp, Artificial Sequence
  Reverse primer for rs1111875
ggtggtctcgagcgtaccatcaagtcatttcctct

SEQ ID NO: 119 - DNA, 31 bp, Artificial Sequence
  Forward primer for rs4712523
ggtggtgaattcttctccttctgttgcaccc

SEQ ID NO: 120 - DNA, 49 bp, Artificial Sequence
  Reverse primer for rs4712523
tgcacgggatatcatcacgtgtaaatctttacatttgggtataaaggat

SEQ ID NO: 121 - DNA, 45 bp, Artificial Sequence
  Forward primer for rs13266634
cgtgatgatatcccgtgcactgatgctttatcaacagcagccagc

SEQ ID NO: 122 - DNA, 43 bp, Artificial Sequence
  Reverse primer for rs13266634
aggtgttttagtttactgcttgttcgaaccacttggctgtccc

SEQ ID NO: 123 - DNA, 44 bp, Artificial Sequence
  Forward primer for rs10012946
gaacaagcagtaaactaaaacaccttggctcaagtgctcactca

SEQ ID NO: 124 - DNA, 44 bp, Artificial Sequence
  Reverse primer for rs10012946
ccgataaggaggctcgaatggcagaataccctctggtttattca

SEQ ID NO: 125 - DNA, 45 bp, Artificial Sequence
  Forward primer for rs2383208
cattcgagcctccttatcggagaaactgtgacaggaaggaagtcc

SEQ ID NO: 126 - DNA, 39 bp, Artificial Sequence
  Reverse primer for rs2383208
ggtggtctcgagttgaaactagtagatgctcaattcatg

SEQ ID NO: 127 - DNA, 41 bp, Artificial Sequence; identical to SEQ ID NO: 109
  Forward primer for rs7903146

SEQ ID NO: 128 - DNA, 50 bp, Artificial Sequence; identical to SEQ ID NO: 110
  Reverse primer for rs7903146

SEQ ID NO: 129 - DNA, 48 bp, Artificial Sequence; identical to SEQ ID NO: 111
  Forward primer for rs1801282

SEQ ID NO: 130 - DNA, 44 bp, Artificial Sequence; identical to SEQ ID NO: 112
  Reverse primer for rs1801282

SEQ ID NO: 131 - DNA, 41 bp, Artificial Sequence; identical to SEQ ID NO: 113
  Forward primer for rs5219

SEQ ID NO: 132 - DNA, 34 bp, Artificial Sequence; identical to SEQ ID NO: 114
  Reverse primer for rs5219

SEQ ID NO: 133 - DNA, 50 bp, Artificial Sequence; identical to SEQ ID NO: 115
  Forward primer for rs4402960

SEQ ID NO: 134 - DNA, 45 bp, Artificial Sequence; identical to SEQ ID NO: 116
  Reverse primer for rs4402960

SEQ ID NO: 135 - DNA, 44 bp, Artificial Sequence; identical to SEQ ID NO: 117
  Forward primer for rs111875

SEQ ID NO: 136 - DNA, 48 bp, Artificial Sequence
  Reverse primer for rs111875
ctgggatcgattaagtaagttgaaccgtaccatcaagtcatttcctct

SEQ ID NO: 137 - DNA, 44 bp, Artificial Sequence
  Forward primer for rs4712523
gttcaacttacttaatcgatcccagttctccttctgttgcaccc

SEQ ID NO: 138 - DNA, 49 bp, Artificial Sequence; identical to SEQ ID NO: 120
  Reverse primer for rs4712523

SEQ ID NO: 139 - DNA, 45 bp, Artificial Sequence; identical to SEQ ID NO: 121
  Forward primer for rs13266634

SEQ ID NO: 140 - DNA, 43 bp, Artificial Sequence; identical to SEQ ID NO: 122
  Reverse primer for rs13266634

SEQ ID NO: 141 - DNA, 44 bp, Artificial Sequence; identical to SEQ ID NO: 123
  Forward primer for rs10012946

SEQ ID NO: 142 - DNA, 44 bp, Artificial Sequence; identical to SEQ ID NO: 124
  Reverse primer for rs10012946

SEQ ID NO: 143 - DNA, 45 bp, Artificial Sequence; identical to SEQ ID NO: 125
  Forward primer for rs2383208

SEQ ID NO: 144 - DNA, 39 bp, Artificial Sequence; identical to SEQ ID NO: 126
  Reverse primer for rs2383208

SEQ ID NO: 145 - DNA, 37 bp, Artificial Sequence
  Forward primer for rs6025
ggtggtagatcttcaaggacaaaatacctgtattcct

SEQ ID NO: 146 - DNA, 40 bp, Artificial Sequence
  Reverse primer for rs6025
acgggttcaaatgtgggtataagcagatccctggacaggc

SEQ ID NO: 147 - DNA, 44 bp, Artificial Sequence
  Forward primer for rs4948418
ttatacccacatttgaacccgttcgcctctggcatgacagggaa

SEQ ID NO: 148 - DNA, 41 bp, Artificial Sequence
  Reverse primer for rs4948418
ttccgctgagacttgactttatttgctgacttacctcagcc

SEQ ID NO: 149 - DNA, 45 bp, Artificial Sequence
  Forward primer for rs2383207
ataaagtcaagtctcagcggaagccactcctgttcggatcccttc

SEQ ID NO: 150 - DNA, 41 bp, Artificial Sequence
  Reverse primer for rs2383207
ggtggtctcgaggctgaaaatagtaaataatcatgcttagc

SEQ ID NO: 151 - DNA, 34 bp, Artificial Sequence
  Forward primer for rs6983267
ggtggtgaattcctttgagctcagcagatgaaag

SEQ ID NO: 152 - DNA, 52 bp, Artificial Sequence
  Reverse primer for rs6983267
ctagagccagtatgtctcatgcacataaaaattctttgtacttttctcagtg

SEQ ID NO: 153 - DNA, 46 bp, Artificial Sequence
  Forward primer for rs4939827
gcatgagacatactggctctagcacagcctcatccaaaagaggaaa

SEQ ID NO: 154 - DNA, 39 bp, Artificial Sequence
  Reverse primer for rs4939827
catgagaagtaggtctcacacggggagctctggggtcct

SEQ ID NO: 155 - DNA, 48 bp, Artificial Sequence
  Forward primer for rs3802842
cgtgtgagacctacttctcatgcgtccttgcagacccatagaaaatct

SEQ ID NO: 156 - DNA, 39 bp, Artificial Sequence
  Reverse primer for rs3802842
ggtggtgcggccgccctaaaatgaggtgaatttctggga

SEQ ID NO: 157 - DNA, 32 bp, Artificial Sequence
  Forward primer for Rs2165241
ggtggtgaattcctgagctctcaaatgccaca

SEQ ID NO: 158 - DNA, 34 bp, Artificial Sequence
  Reverse primer for Rs2165241
cctgtcccacaccacctacccaggcatgcctctg

SEQ ID NO: 159 - DNA, 47 bp, Artificial Sequence
  Forward primer for Rs1219648
taggtggtgtgggacaggacgttcgagcacgcctattttacttgaca

SEQ ID NO: 160 - DNA, 41 bp, Artificial Sequence
  Reverse primer for Rs1219648
gctgtagaaaaccgaaggatacggccatggccatccttgaa

SEQ ID NO: 161 - DNA, 48 bp, Artificial Sequence
  Forward primer for Rs3803662
cgtatccttcggttttctacagctcagtccacagttttattcttcgct

SEQ ID NO: 162 - DNA, 47 bp, Artificial Sequence
  Reverse primer for Rs3803662
cctgtacggttcttatccgagtatctctccttaatgcctctatagct

SEQ ID NO: 163 - DNA, 49 bp, Artificial Sequence
  Forward primer for Rs8034191
tactcggataagaaccgtacaggacagcccaatgtggtataagttttct

SEQ ID NO: 164 - DNA, 36 bp, Artificial Sequence
  Reverse primer for Rs8034191
ggtggtgcggccgcagttactatctgtcagggcctt

SEQ ID NO: 165 - DNA, 38 bp, Artificial Sequence
  Forward primer for Rs9888739
ggtggtagatctagtatgcagaactcactatgttgtaa

SEQ ID NO: 166 - DNA, 49 bp, Artificial Sequence
  Reverse primer for Rs9888739
ggcacggtgatgtggcagtcaaagaggttctatatttttatcattacag

SEQ ID NO: 167 - DNA, 50 bp, Artificial Sequence
  Forward primer for Rs7574865
gccacatcaccgtgcccgcggatctaagtatgaaaagttggtgaccaaaa

SEQ ID NO: 168 - DNA, 48 bp, Artificial Sequence
  Reverse primer for Rs7574865
gacagtagccatcttccagggaaattccactgaaataagataaccact

SEQ ID NO: 169 - DNA, 49 bp, Artificial Sequence
  Forward primer for Rs2187668
cctggaagatggctactgtctcgtgaacaatcattttaccacatggtcc

SEQ ID NO: 170 - DNA, 44 bp, Artificial Sequence
  Reverse primer for Rs2187668
gatttccctcagttgtgtagacacacatatgaggcagctgagag

SEQ ID NO: 171 - DNA, 46 bp, Artificial Sequence
  Forward primer for Rs10488631
tgtctacacaactgagggaaatcaaggctgcttccatagctagtct

SEQ ID NO: 172 - DNA, 32 bp, Artificial Sequence
  Reverse primer for Rs10488631
ggtggtctcgaggccttgtagctcggaaatgg

SEQ ID NO: 173 - DNA, 35 bp, Artificial Sequence
  Forward primer for rs6897932
ggtggtgaattcaggggagatggatcctatcttac

SEQ ID NO: 174 - DNA, 42 bp, Artificial Sequence
  Reverse primer for rs6897932
aatgggccattcggctcgacagagaaaaaactcaaaatgctg

SEQ ID NO: 175 - DNA, 49 bp, Artificial Sequence
  Forward primer for rs3135388
gagccgaatggcccattgggtaatcgtcctcatcaggaaaacctaaagt

SEQ ID NO: 176 - DNA, 47 bp, Artificial Sequence
  Reverse primer for rs3135388
gactgtactttagggtaagcagattcagtagagatctcccaacaaac

SEQ ID NO: 177 - DNA, 47 bp, Artificial Sequence
  Forward primer for rs3751812
atctgcttaccctaaagtacagtccacctgaaaataggtgagctgtc

SEQ ID NO: 178 - DNA, 28 bp, Artificial Sequence
  Reverse primer for rs3751812
ggtggtctcgaggagcctctccctgcca

SEQ ID NO: 179 - DNA, 38 bp, Artificial Sequence
  Forward primer for rs2395185
ggtggtagatctactactacactacatgaagccaaaaa

SEQ ID NO: 180 - DNA, 45 bp, Artificial Sequence
  Reverse primer for rs2395185
ctgggatcgattaagtaagttgaacacagcagaattctccaggga

SEQ ID NO: 181 - DNA, 43 bp, Artificial Sequence
  Forward primer for rs9858542
gttcaacttacttaatcgatcccagcgagcaagctggcaaact

SEQ ID NO: 182 - DNA, 42 bp, Artificial Sequence
  Reverse primer for rs9858542
ctacgaccgatcgcaatcagcaaaatgcaggcagtgcatacc

SEQ ID NO: 183 - DNA, 46 bp, Artificial Sequence
  Forward primer for rs10883365
tgattgcgatcggtcgtagaggtagttcgttctcagacggtttgaa

SEQ ID NO: 184 - DNA, 41 bp, Artificial Sequence
  Reverse primer for rs10883365
cctggcccttcagaggtatcgcacaggggtcacgttggcac

SEQ ID NO: 185 - DNA, 57 bp, Artificial Sequence
  Forward primer for rs11209026
gatacctctgaagggccaggatcagttctttgattgggatatttaacagatcatcat

SEQ ID NO: 186 - DNA, 35 bp, Artificial Sequence; identical to SEQ ID NO: 42
  Reverse primer for rs11209026

SEQ ID NO: 187 - DNA, 28 bp, Artificial Sequence
  Forward primer for rs671
ggtggtagatctcgggctgcaggcatac

SEQ ID NO: 188 - DNA, 37 bp, Artificial Sequence
  Reverse primer for rs671
ctgttttgcgctcgcggtcccacactcacagttttca

SEQ ID NO: 189 - DNA, 46 bp, Artificial Sequence
  Forward primer for rs713598
cgcgagcgcaaaacagcgcttggacgcacacaatcactgttgctca

SEQ ID NO: 190 - DNA, 45 bp, Artificial Sequence
  Reverse primer for rs713598
ccaatggaaaagctgcaggagaatttttgggatgtagtgaagagg

SEQ ID NO: 191 - DNA, 45 bp, Artificial Sequence
  Forward primer for rs17822931
tcctgcagcttttccattggctagcaccaagtctgccacttactg

SEQ ID NO: 192 - DNA, 32 bp, Artificial Sequence
  Reverse primer for rs17822931
ggtggtctcgaggcttctgcattgccagtgta

SEQ ID NO: 193 - DNA, 35 bp, Artificial Sequence
  Forward primer for rs12913832
ggtggtagatctggccagtttcatttgagcattaa

SEQ ID NO: 194 - DNA, 41 bp, Artificial Sequence
  Reverse primer for rs12913832
agaggtaattccttgtgtgcattagcgtgcagaacttgaca

SEQ ID NO: 195 - DNA, 43 bp, Artificial Sequence
  Forward primer for rs4988235
aatgcacacaaggaattacctcttcgttcctttgaggccaggg

SEQ ID NO: 196 - DNA, 46 bp, Artificial Sequence
  Reverse primer for rs4988235
cgccggacaaaagtactctgctggcaatacagataagataatgtag

SEQ ID NO: 197 - DNA, 48 bp, Artificial Sequence
  Forward primer for rs2814778
agagtacttttgtccggcggcgtcaccctcattagtccttggctctta

SEQ ID NO: 198 - DNA, 39 bp, Artificial Sequence
  Reverse primer for rs2814778
tgcctaacctccttaatcggatgcgcctgtgcttccaag

SEQ ID NO: 199 - DNA, 41 bp, Artificial Sequence
  Forward primer for rs1815739
atccgattaaggaggttaggcaggcactgcccgaggctgac

SEQ ID NO: 200 - DNA, 29 bp, Artificial Sequence
  Reverse primer for rs1815739
ggtggtctcgaggatggcacctcgctctc

SEQ ID NO: 201 - DNA, 27 bp, Artificial Sequence
  Forward primer for rs601338
ggtggtagatctccggctacccctgct

SEQ ID NO: 202 - DNA, 37 bp, Artificial Sequence
  Reverse primer for rs601338
gcccatattccagggcccgcggaggtggtggtagaag

SEQ ID NO: 203 - DNA, 54 bp, Artificial Sequence
  Forward primer for rs3923809
gggccctggaatatgggcaacatgcagtgaaaataaaatgatagctttctctct

SEQ ID NO: 204 - DNA, 35 bp, Artificial Sequence
  Reverse primer for rs3923809
ggtggtctcgaggtcctactgaattgcagatggat

SEQ ID NO: 205 - DNA, 50 bp, Artificial Sequence
  Forward primer (Mutant Site Exon 18)
atctcgatcccgcgaaattaatacgagatctgtggagaagctcccaacca

SEQ ID NO: 206 - DNA, 44 bp, Artificial Sequence
  Reverse primer (Mutant Site Exon 18)
ctacgaccgatcgcaatcagcaaaacttatacaccgtgccgaac

SEQ ID NO: 207 - DNA, 54 bp, Artificial Sequence
  Forward primer (Mutant Site Exon 19)
tgattgcgatcggtcgtagaggtagaggtaaaagttaaaattcccgtcgctatc

SEQ ID NO: 208 - DNA, 44 bp, Artificial Sequence
  Reverse primer (Mutant Site Exon 19)
ctgggatcgattaagtaagttgaacccttgttggctttcggaga

SEQ ID NO: 209 - DNA, 49 bp, Artificial Sequence
  Forward primer (Mutant Site Exon 21)
gttcaacttacttaatcgatcccaggcatgtcaagatcacagattttgg

SEQ ID NO: 210 - DNA, 45 bp, Artificial Sequence
  Reverse primer (Mutant Site Exon 21)
cctggcccttcagaggtatctgcatggtattctttctcttccgca

SEQ ID NO: 211 - DNA, 39 bp, Artificial Sequence
  Forward primer (Mutant Site Exon 20)
gatacctctgaagggccaggcatctgcctcacctccacc

SEQ ID NO: 212 - DNA, 45 bp, Artificial Sequence
  Reverse primer (Mutant Site Exon 20)
gcacgatgccggtgaacgcggccgccaccagttgagcaggtactg

SEQ ID NO: 213 - DNA, 25 bp, Artificial Sequence
  Forward primer for detecting EGFR mutation
atctcgatcccgcgaaattaatacg

SEQ ID NO: 214 - DNA, 17 bp, Artificial Sequence
  Reverse primer for detecting EGFR mutation
gcacgatgccggtgaac

SEQ ID NO: 215 - DNA, 23 bp, Artificial Sequence
  primer for Sequencing
gaagaaggtaaactgacaaatcc

SEQ ID NO: 216 - DNA, 15 bp, Artificial Sequence
  Exon 19 deletion mutation
gaattaagagaagca